(19) United States
(12) Patent Application Publication
(10) Pub. No.: US 2009/0202569 A1
(43) Pub. Date: Aug. 13, 2009

(54) Prophylactic/Therapeutic Agent for Cancer

(76) Inventors: Tetsuo Mashima, Tokyo (JP); Takashi Tsuruo, Tokyo (JP); Hideaki Tojo, Ibaraki (JP); Hiroyuki Sumi, Osaka (JP)

Correspondence Address: FOLEY AND LARDNER LLP;SUITE 500, 3000 K STREET NW, WASHINGTON, DC, 20007, US

(21) Appl. No.: 12/225,882

(22) PCT Filed: Apr. 6, 2007
(86) PCT No.: PCT/JP2007/058125

§ 371 Date: Nov. 10, 2008

(30) Foreign Application Priority Data
Apr. 7, 2006 (JP) 2006-106928

(51) Int. Cl. A61K 39/395; C07K 16/18; C07H 21/02; A61K 31/7088; C12N 5/06

(52) U.S. Cl. 424/174.1; 530/387.1; 536/24.5; 435/375

(57) ABSTRACT

The present invention provides a preventative/therapeutic agent for cancer capable of selectively and effectively killing cancer cells. A medicament of the present invention prepared by combining at least one selected from a substance that inhibits the activity of an enzyme belonging to the acyl-CoA synthase family and a substance that inhibits expression of a gene for an enzyme belonging to the acyl-CoA synthase family, with at least one selected from a substance that inhibits the activity of fatty acid synthase and a substance that inhibits expression of a fatty acid synthase gene and the like can be used as a preventative/therapeutic agent for cancer capable of selectively and effectively killing cancer cells.

### TECHNICAL FIELD

The present invention relates to a preventative/therapeutic agent for cancer.

### BACKGROUND OF THE INVENTION

Fatty acid synthase (FAS) is an enzyme responsible for conversion of malonyl-CoA into long-chain fatty acids, which is an early reaction in fatty acid biosynthesis (Wakil, S. J., Biochemistry, Vol. 28, p. 4523-4530, 1989). FAS is overexpressed in many cancer cells (Kuhajda, F. P. et al., Proc. Natl. Acad. Sci. USA, Vol. 91, p. 6379-6383, 1994). Inhibition of FAS expression or FAS activity selectivity suppresses proliferation and induces cell death of cancer cells, with little toxicity towards normal cells (Kuhajda, F. P. et al., Proc. Natl. Acad. Sci. USA, Vol. 97, p. 3450-3454, 2000; De Schrijver, E. et al., Cancer Res., Vol. 63, p. 3799-3804, 2003). Known FAS inhibitors include Cerulenin, a natural low-molecular-weight compound derived from Cephalosporium caerulence (Vance, D. et al., Biochem. Biophys. Res. Commun., Vol. 48, p. 649-656, 1972) and the synthetic low-molecular weight compound C75 (Pizer, E. S. et al., Cancer Res., Vol. 60, p. 213-218, 2000).

On the other hand, enzymes in the acyl-CoA synthase (hereunder abbreviated as ACS) family are responsible for converting long-chain fatty acids into acyl-CoA. Because acyl-CoA is a substrate for intercellular lipid synthesis and fatty acid degradation/elongation reactions, ACS play a central role in intercellular lipid metabolism and also in intercellular signaling by means of lipids. Enzymes in ACS family are also involved in extracellular fatty acid uptake (Faergemen, N. J. & Knudsen, J., Biochemical J., Vol. 323, p. 1-12, 1997). Five isozymes (ACS1, 3, 4, 5, 6) with different substrate specificities and intracellular locations have already been identified in humans and rodents (Coleman, R. A. et al., J. Nutr., Vol. 132, p. 2123-2126, 2002). Of these, ACS4 and ACS5 are overexpressed in human colon cancer and human gliomas (Cao, Y. et al., Cancer Res., Vol. 61, p. 8429-8434, 2001; Yamashita, Y. et al., Oncogene, Vol. 19, p. 5919-5925, 2000). ACS inhibition also induces cancer-specific cell death (Mashima, T. et al., J. Natl. Cancer Inst., Vol. 97, p. 765-777, 2005). Triacsin C, a natural low-molecular-weight compound derived from Streptomyces sp., is known as an ACS inhibitor (Tomoda, H. et al., Biochem. Biophys. Acta, Vol. 921, p. 595-598, 1987). Triacsin C is known to inhibit ACS1 and ACS4 (Kim, J.-H. et al., J. Biol. Chem., Vol. 276, p. 24667-24673, 2001).

### DISCLOSURE OF THE INVENTION

By means of the current state of cancer therapy, many cancers cannot be cured with existing methods and there is demand for development of new therapies. A particular problem is the side-effects produced by existing anti-cancer agents in normal human tissue. There is strong demand for development of safe and advanced method which is selectively and powerfully inducing cancer cell death however showing low toxicity in normal cells in order to improve therapeutic results against cancer.

As a result of exhaustive research aimed at solving these problems, the inventors discovered that cancer cells could be killed more forcefully by simultaneously inhibiting both ACS activity and FAS activity. The present invention was perfected as a result of further research based on this finding.

That is, the present invention provides, such as:

[1] A preventative/therapeutic agent for cancer prepared by combining (i) at least one selected from a substance that inhibits the activity of an enzyme belonging to the acyl-CoA synthase family and a substance that inhibits expression of a gene for an enzyme belonging to the acyl-CoA synthase family, with (ii) at least one selected from a substance that inhibits the activity of fatty acid synthase and a substance that inhibits expression of a fatty acid synthase gene,

[2] The preventative/therapeutic agent according to [1] above, wherein the enzyme belonging to the acyl-CoA synthase family is at least one selected from the proteins comprising the same or substantially the same amino acid sequences represented by SEQ ID NO:1, SEQ ID NO:3, SEQ ID NO:5, SEQ ID NO:7, SEQ ID NO:9, SEQ ID NO:11, SEQ ID NO:13 and SEQ ID NO:15, or partial peptides or salts thereof,

[3] The preventative/therapeutic agent according to [1] above, wherein the enzyme belonging to the acyl-CoA synthase family is at least one selected from the proteins comprising the same or substantially the same amino acid sequences represented by SEQ ID NO:1, SEQ ID NO:3, SEQ ID NO:5, SEQ ID NO:7, SEQ ID NO:9 and SEQ ID NO:11, or partial peptides or salts thereof,

[4] The preventative/therapeutic agent according to [1] above, wherein the fatty acid synthase is a protein comprising the same or substantially the same amino acid sequence represented by SEQ ID NO:17, or a partial peptide or salt thereof,

[5] The preventative/therapeutic agent according to [1] above, prepared by combining (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family, with (ii) at least one selected from (a) a compound or salt thereof that inhibits the activity of fatty acid synthase, (b) a compound or salt thereof that inhibit expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase,

[6] The preventative/therapeutic agent according to [1] above, prepared by combining a compound or salt thereof that inhibits the activity of an enzyme belonging to the acyl-CoA synthase family with a compound or salt thereof that inhibits the activity of fatty acid synthase,

[7] A preventative/therapeutic agent for cancer comprising (i) a substance that inhibits the activities of an enzyme belonging to the acyl-CoA synthase family and fatty acid synthase, and/or (ii) a substance that inhibits expression of a gene for an enzyme belonging to the acyl-CoA synthase family and expression of a fatty acid synthase gene,

[8] The preventative/therapeutic agent according to Claim 1, wherein the preventative/therapeutic agent for cancer is a combination preparation,

[9] The preventative/therapeutic agent according to Claim 1, wherein the preventative/therapeutic agent is a kit comprising (i) a medicament comprising at least one selected from a substance that inhibits the activity of an enzyme belonging to the acyl-CoA family and a substance that inhibits expression of a gene for an enzyme belonging to the acyl-CoA family and (ii) a medicament comprising at least one selected from a substance that inhibits the activity of fatty acid synthase and a substance that inhibits expression of a fatty acid synthase gene,

[10] A method for preventing/treating cancer wherein (i) the activity of an enzyme belonging to the acyl-CoA family and/or expression of a gene for the enzyme is inhibited and (ii) the activity of fatty acid synthase and/or expression of a gene for the enzyme is inhibited,

[11] A method for preventing/treating cancer wherein (i) an effective dose of at least one selected from a substance that inhibits the activity of an enzyme belonging to the acyl-CoA synthase family and a substance that inhibits expression of a gene for an enzyme belonging to the acyl-CoA synthase family, and (ii) an effective dose of at least one selected from a substance that inhibits the activity of fatty acid synthase and a substance that inhibits expression of a fatty acid synthase gene, are administered to a mammal,

[12] A use of (i) at least one selected from a substance that inhibits the activity of an enzyme belonging to the acyl-CoA synthase family and a substance that inhibits expression of a gene for an enzyme belonging to the acyl-CoA synthase family, and (ii) at least one selected from a substance that inhibits the activity of fatty acid synthase and a substance that inhibits expression of a fatty acid synthase gene, to manufacture a preventative/therapeutic agent for cancer,

[13] A cancer cell apoptosis promoting agent or cancer cell proliferation inhibiting agent prepared by combining (i) at least one selected from a substance that inhibits the activity of an enzyme belonging to the acyl-CoA synthase family and a substance that inhibits expression of a gene for an enzyme belonging to the acyl-CoA synthase family, with (ii) at least one selected from a substance that inhibits the activity of fatty acid synthase and a substance that inhibits expression of a fatty acid synthase gene,

[14] The agent according to [13] above, prepared by combining (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family, with (ii) at least one selected from (a) a compound or salt thereof that inhibits the activity of fatty acid synthase, (b) a compound or salt thereof that inhibits expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase,

[15] The agent according to [13] above, prepared by combining a compound or salt thereof that inhibits the activity of an enzyme belonging to the acyl-CoA synthase family with a compound or salt thereof that inhibits the activity of fatty acid synthase,

[16] A cancer cell apoptosis promoting agent or cancer cell proliferation inhibiting agent, comprising (i) a substance that inhibits the activities of an enzyme belonging to the acyl-CoA synthase family and fatty acid synthase, and/or (ii) a substance that inhibits expression of a gene for an enzyme belonging to the acyl-CoA synthase family and expression of a fatty acid synthase gene,

[16′] A cancer cell apoptosis promotion method or cancer cell proliferation inhibition method wherein (i) the activities of an enzyme belonging to the acyl-CoA synthase family and fatty acid synthase are inhibited, and/or (ii) expression of a gene for an enzyme belonging to the acyl-CoA synthase family and expression of a fatty acid synthase gene are inhibited,

[16″] A cancer cell apoptosis promotion method or cancer cell proliferation inhibition method wherein (i) an effective dose of a substance that inhibits the activities of an enzyme belonging to the acyl-CoA synthase family and fatty acid synthase, and/or (ii) an effective dose of a substance that inhibits expression of a gene for an enzyme belonging to the acyl-CoA synthase family and expression of a fatty acid synthase gene, are administered to a mammal,

[17] A cancer cell apoptosis promotion method or cancer cell proliferation inhibition method wherein (i) the activity of an enzyme belonging to the acyl-CoA synthase family and/or expression of a gene for the enzyme is inhibited, and (ii) the activity of fatty acid synthase and/or expression of a gene for the enzyme is inhibited.

[17′] A cancer cell apoptosis promotion method or cancer cell proliferation inhibition method wherein (i) an effective dose of a substance that inhibits the activity of an enzyme belonging to the acyl-CoA synthase family and/or expression of a gene for the enzyme and (ii) an effective dose of a substance that inhibits the activity of fatty acid synthase and/or expression of a gene for the enzyme are administered to a mammal.

A “preventative/therapeutic agent for cancer” may itself be a substance having a preventative/therapeutic effect against cancer (such as a synthetic compound, peptide, protein, antibody, nonpeptidal compound, fermentation product, cell extract, plant extract, animal tissue extract, serum or the like), or may be a medicament containing such a substance. “Cancer prevention” includes prevention of metastasis and/or reoccurrence of cancer.

A “cancer cell apoptosis promoting medicament” may itself be a substance having a cancer cell apoptosis-promoting effect, or may be a medicament containing such a substance (such as a synthetic compound, peptide, protein, antibody, noneptidal compound, fermentation product, cell extract, plant extract, animal tissue extract, serum or the like).

A “cancer cell proliferation inhibiting medicament” may itself be a substance having a cancer cell proliferation inhibition effect (such as a synthetic compound, peptide, protein, antibody, noneptidal compound, fermentation product, cell extract, plant extract, animal tissue extract, serum or the like), or may be a medicament containing such a substance.

“Inhibiting expression of a gene” means that production of the protein coded for by that gene is inhibited by inhibiting any of the series of events (including transcription (mRNA production) and translation (protein production) for example) leading from the gene to protein production.

“Inhibiting expression of a protein” means that production of the protein is inhibited by inhibiting any of the series of events leading from the gene coding for the protein to protein production (including transcription (mRNA production) and translation (protein production) for example).

“Low-molecular-weight compounds” are organic and inorganic compounds with molecular weights of 10,000 or less (preferably 5,000 or less, more preferably 2,000 or less, still more preferably 700 or less).

### BRIEF DESCRIPTION OF THE DRAWINGS

FIG. 1 shows the results for inhibition of cell proliferation when the FAS inhibitor Cerulenin (15 μg/ml) or C75 (15 μg/ml) was added to SF268/mock or SF268/ACS5 cells and cultured for 24 hours. The cell survival rate (%) using 100% untreated SF268/mock is shown on the vertical axis.

The enzyme belonging to the acyl-CoA synthase family and the fatty acid synthase (sometimes abbreviated below as proteins used in the present invention) may be synthetic proteins or proteins derived from the cells (for example, retinal cells, liver cells, spleen cells, nerve cells, glial cells, pancreatic cells, bone marrow cells, mesangial cells, Langerhans cells, epidermal cells, epithelial cells, endothelial cells, fibroblasts, fiber cells, muscle cells, fat cells, immune cells (such as macrophages, T-cells, B-cells, natural killer cells, mast cells, neutrophils, basophils, eosinophils, monocytes, platelets, etc.), megakaryocytes, synovial cells, cartilage cells, bone cells, osteoblasts, osteoclasts, mammary cells, liver cells or mesenchymal cells, or precursor cells, stem cells or cancer cells of these) of humans or warm-blooded animals (for example, guinea pigs, rats, mice, chickens, rabbits, pigs, sheep, cows and monkeys) or from tissues in which these cells are present, such as the brain, various parts of the brain (for example the retina, olfactory bulb, amygdaloid nucleus, basal ganglia, hippocampus, thalamus, hypothalamus, cerebral cortex, medulla oblongata and cerebellum), spinal cord, pituitary gland, stomach, pancreas, kidneys, liver, reproductive glands, thyroid gland, gallbladder, bone marrow, adrenal gland, skin, muscle, lungs, digestive tract (for example the large intestine and small intestine), blood vessels, heart, thymus, spleen, submandibular gland, peripheral blood, prostate gland, testicles, ovaries, placenta, uterus, bone, joints, skeletal muscle and the like, or from blood cells or cultured cells thereof (for example, MEL, M1, CTLL-2, HT-2, WEHI-3, HL-60, JOSK-1, K562, ML-1, MOLT-3, MOLT-4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01 and the like).

The enzyme belonging to the acyl-CoA synthase family may be at least one selected from the proteins containing the same or substantially the same amino acid sequences represented by SEQ ID NO:1, SEQ ID NO:3, SEQ ID NO:5, SEQ ID NO:7, SEQ ID NO:9, SEQ ID NO:11, SEQ ID NO:13 and SEQ ID NO:15, or the partial peptides or salts thereof.

The fatty acid synthase may be at least one selected from the proteins containing the same or substantially the same amino acid sequence represented by SEQ ID NO:17, or the partial peptides or salts thereof.

The same or substantially the same amino acid sequence represented by SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17 may be an amino acid sequence having about 50% or greater or preferably about 60% or greater or more preferably about 70% or greater or still more preferably about 80% or greater or especially about 90% or greater or ideally about 95% or greater homology with an amino acid sequence represented by SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

Amino acid sequence homology may be calculated using the homology calculation algorithm NCBI BLAST (National Center for Biotechnology Information Basic Local Alignment Search Tool), under the following conditions: expected value=10; gaps allowed; matrix=BLOSUM62; filtering OFF.

A protein containing substantially the same amino acid sequence represented by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 is for example preferably a protein that contains substantially the same amino acid sequence represented by the aforementioned SEQ ID NO:1, SEQ ID NO:3, SEQ ID NO:5, SEQ ID NO:7, SEQ ID NO:9, SEQ ID NO:11, SEQ ID NO:13 or SEQ ID NO:15, and that has substantially the same activity as a protein containing an amino acid sequence represented by SEQ ID NO:1, SEQ ID NO:3, SEQ ID NO:5, SEQ ID NO: 7, SEQ ID NO:9, SEQ ID NO:11, SEQ ID NO:13 or SEQ ID NO:15.

The activity that is substantially the same may be acyl-CoA synthase activity for example. Substantially the same means that the properties are of the same kind (that is, physiologically or pharmacologically the same). Thus, preferably the acyl-CoA synthase activity is equivalent (such as about 0.01 to 100 times or preferably about 0.1 to 10 times or more preferably 0.5 to 2 times), but quantitative factors such as the degree of such activity and the molecular weight of the protein may be different.

Acyl-CoA synthase activity can be measured by known methods, such as the methods described in J. Biol. Chem., Vol. 256, p. 5702-5707, 1981 or equivalent methods. Specifically, the protein used in the present invention is reacted for 10 minutes at 35° C. in 0.5 ml of solution containing 0.2 M Tris-HCl buffer (pH 7.5), 2.5 mM ATP, 8 mM MgCl₂, 2 mM EDTA, 20 mM NaF, 0.1% (w/v) Triton X-100, 10 μM [1-¹⁴C] palmitic acid (5 μCi/μmol) and 0.5 mM coenzyme A (CoA). The reaction begins when the CoA is added, and is stopped by adding 2.5 ml of isopropanol:n-heptane:1 M sulfuric acid (40:10:1 v/v). After the reaction has been stopped, 0.5 ml of water and 2.5 ml of n-heptane are added to remove an organic solvent layer containing unreacted fatty acids, the water layer is washed three times with 2.5 ml n-heptane, and the radioactivity remaining in the water layer is measured with a scintillation counter.

The protein containing substantially the same amino acid sequence represented by SEQ ID NO:17 is for example preferably a protein that contains substantially the same amino acid sequence represented by the aforementioned SEQ ID NO:17, and that has substantially the same activity as a protein containing the amino acid sequence represented by SEQ ID NO:17.

The activity that is substantially the same may be fatty acid synthase activity for example. Substantially the same means that the properties are of the same kind (that is, physiologically or pharmacologically the same). Thus, preferably the fatty acid synthase activity is equivalent (such as about 0.01 to 100 times or preferably about 0.1 to 10 times or more preferably 0.5 to 2 times), but quantitative factors such as the degree of such activity and the molecular weight of the protein may be different.

Fatty acid synthase activity can be measured by known methods, such as those described in Nepokroeff, C. M. et al., Methods Enzymol., Vol. 26, p. 37-39, 1975 or equivalent methods. Specifically, the protein used in the present invention is reacted for 5 minutes at 30° C. in 1 ml of reaction solution containing 0.5 M potassium phosphate buffer (pH 7.0), 0.2 mM malonyl-CoA, 0.066 mM acetyl-CoA and 0.2 mM NADPH, absorbancy at 340 nm is measured before and after the reaction, and enzyme activity is calculated from the difference between the two absorbancy values.

A protein used in the present invention may also be for example (1) a so-called mutein such as a protein containing (i) the amino acid sequence represented by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 with 1 or 2 or more (such as about 1 to 100 or preferably 1 to 30 or more preferably 1 to 10 or still more preferably a few (1 to 5)) amino acids deleted therefrom, (ii) the amino acid sequence represented by SEQ ID NO:1, SEQ ID NO:3, SEQ ID NO:5, SEQ ID NO:7, SEQ ID NO:9, SEQ ID NO:1, SEQ ID NO:13 or SEQ ID NO:15 with 1 or 2 or more (such as about 1 to 100 or preferably 1 to 30 or more preferably 1 to 10 or still more preferably a few (1 to 5)) amino acids added thereto, (iii) the amino acid sequence represented by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 with 1 or 2 or more (such as about 1 to 100 or preferably 1 to 30 or more preferably 1 to 10 or still more preferably a few (1 to 5)) amino acids inserted therein, (iv) the amino acid sequence represented by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 with 1 or 2 or more (such as about 1 to 100 or preferably 1 to 30 or more preferably 1 to 10 or still more preferably a few (1 to 5)) amino acids replaced with other amino acids or (v) an amino acid sequence consisting of a combination of these, (2) a so-called mutein such as a protein containing (i) the amino acid sequence represented by SEQ ID NO:17 with 1 or 2 or more (such as about 1 to 100 or preferably 1 to 30 or more preferably 1 to 10 or still more preferably a few (1 to 5)) amino acids deleted therefrom, (ii) the amino acid sequence represented by SEQ ID NO:17 with 1 or 2 or more (such as about 1 to 100 or preferably 1 to 30 or more preferably 1 to 10 or still more preferably a few (1 to 5)) amino acids added thereto, (iii) the amino acid sequence represented by SEQ ID NO:17 with 1 or 2 or more (such as about 1 to 100 or preferably 1 to 30 or more preferably 1 to 10 or still more preferably a few (1 to 5)) amino acids inserted therein, (iv) the amino acid sequence represented by SEQ ID NO:17 with 1 or 2 or more (such as about 1 to 100 or preferably 1 to 30 or more preferably 1 to 10 or still more preferably a few (1 to 5)) amino acids replaced with other amino acids or (v) an amino acid sequence consisting of a combination of these.

When there are insertions, deletions or substitutions in an amino acid sequence as described above, the locations of such insertions, deletions or substitutions are not particularly limited.

In this Description, the left end of a protein is called the N-terminal (amino terminal) and the right end is called the C-terminal (carboxyl terminal) in accordance with the rules of peptide nomenclature. In proteins containing the amino acid sequence represented by SEQ ID NO:1 and other proteins used in the present invention, the C-terminal may be either a carboxyl group (—COOH), carboxylate (—COO⁻), amide (—CONH₂) or ester (—COOR).

In this case, a methyl, ethyl, n-propyl, isopropyl, n-butyl or other C₁₋₆ alkyl group for example, a cyclopentyl, cyclohexyl or other C₃₋₈ cycloalkyl group for example, a phenyl, α-naphthyl or other C₆₋₁₂ aryl group for example, a benzyl, phenethyl or other phenyl-C₁₋₂ alkyl group for example or an α-naphthylmethyl or other α-naphthyl-C₁₋₂ alkyl group or other C₇₋₁₄ aralkyl group or pivaloyloxymethyl group or the like for example can be used as the R in the ester.

When a protein used in the present invention has a carboxyl group (or carboxylate) in a position other than the C-terminal, it is included as a protein used in the present invention if the carboxyl group is amidated or esterified. The ester in this case may be one such as the C-terminal ester described above or the like.

A protein used in the present invention may also be one in which an amino group of an amino acid residue (methionine residue for example) of the N-terminal is protected with a protective group (such as a formyl group, acetyl group or other C₁₋₆ alkanoyl or other C₁₋₆ acyl group), one in which a glutamine residue of the N-terminal produced by cleavage in vivo is converted to pyroglutamate, one in which a substituent (—OH, —SH, amino group, imidazole group, indole group, guanidino group or the like) on the side chain of an amino acid in the molecule is protected with a suitable protective group (such as a formyl group, acetyl group or other C₁₋₆ alkanoyl or other C₁₋₆ acyl group), or a fused protein such as a so-called glycoprotein having a sugar chain bound thereto.

Specific examples of proteins used in the present invention include proteins containing the amino acid sequence represented by SEQ ID NO:1, proteins containing the amino acid sequence represented by SEQ ID NO:3, proteins containing the amino acid sequence represented by SEQ ID NO:5, proteins containing the amino acid sequence represented by SEQ ID NO:7, proteins containing the amino acid sequence represented by SEQ ID NO:9, proteins containing the amino acid sequence represented by SEQ ID NO:11, proteins containing the amino acid sequence represented by SEQ ID NO:13, proteins containing the amino acid sequence represented by SEQ ID NO:15 and proteins containing the amino acid sequence represented by SEQ ID NO:17 and the like for example.

A partial peptide of a protein used in the present invention is a partial peptide of an aforementioned protein used in the present invention, and may preferably be any having properties equivalent to those of the aforementioned proteins used in the present invention.

For example, a peptide having an amino acid sequence of at least 20 or more or preferably 50 or more or more preferably 70 or more or still more preferably 100 or more or ideally 200 or more amino acids out of the constituent amino acid sequence of a protein used in the present invention can be used.

In a partial peptide used in the present invention, 1 or 2 or more (preferably about 1 to 20 or more preferably 1 to 10 or still more preferably a few (1 to 5)) amino acids may be deleted, 1 or 2 or more (preferably about 1 to 20 or more preferably 1 to 10 or still more preferably a few (1 to 5)) amino acids may be added, 1 or 2 or more (preferably about 1 to 20 or more preferably 1 to 10 or still more preferably a few (1 to 5)) amino acids may be inserted, or 1 or 2 or more (preferably about 1 to 20 or more preferably 1 to 10 or still more preferably a few (1 to 5)) amino acids may be replaced with other amino acids in the amino acid sequence.

Moreover, in a partial peptide used in the present invention the C-terminal may be a carboxyl group (—COOH), carboxylate (—COO⁻), amide (—CONH₂) or ester (—COOR).

Like a protein used in the present invention, moreover, a partial peptide used in the present invention may be one having a carboxyl group (or carboxylate) at a position other than the C-terminal, one in which an amino group of an N-terminal amino acid residue (such as a methionine residue) is protected, one in which a glutamine residue produced by cleavage of the N-terminal in vivo is converted to pyroglutamate, one in which a substituent on the side chain of an amino acid in the molecule is protected with a suitable protective group, or a so-called glycopeptide having a sugar chain bound thereto.

A partial peptide used in the present invention can be used as an antigen for antibody preparation.

A salt with a physiologically acceptable acid (inorganic or organic acid for example) or base (alkali metal salt for example) or the like can be used as a salt of a protein or partial peptide used in the present invention, and a physiologically acceptable acid addition salt is particularly desirable. For example, salts with inorganic acids (such as hydrochloric acid, phosphoric acid, hydrobromic acid and sulfuric acid) or organic acids (such as acetic acid, formic acid, propionic acid, fumaric acid, maleic acid, succinic acid, tartaric acid, citric acid, malic acid, oxalic acid, benzoic acid, methanesulfonic acid and benzenesulfonic acid) and the like can be used as such salts.

A protein or its partial peptide of the present invention, or a salt thereof, can be manufactured by known protein preparation methods from the aforementioned cells or tissues of humans or warm-blooded animals, or can be manufactured by culturing a transformant containing DNA coding for the protein. It may also be manufactured in accordance with the peptide synthesis methods described below.

In the case of manufacture from tissue or cells of humans or mammals, the human or mammal tissue or cell can be homogenized and extracted with acid or the like, and the resulting extract can then be purified and isolated by a combination of reverse-phase chromatography, ion-exchange chromatography and other chromatography methods.

A commercial resin for protein synthesis can normally be used for synthesizing a protein or its partial peptide of the present invention, or a salt or amide thereof. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenyl acetamidomethyl resin, polyacrylamide resin, 4-(2′-4′-dimethoxyphenyl-hydroxymethyl)phenoxy resin, 4-(2′,4′-dimethoxyphenyl-Fmoc aminoethyl)phenoxy resin and the like. Using such a resin, amino acids having the α-amino groups and side-chain functional groups suitably protected are condensed on the resin by various known condensation methods according to the sequence of the target protein. At the end of the reaction, the protein or partial peptide is excised from the resin while the various protective groups are removed at the same time, and an intramolecular disulfide bond-forming reaction is then performed in a highly diluted solution to obtain the target protein or its partial peptide of the present invention, or an amide thereof.

A variety of activating reagents used in protein synthesis can be used when condensing the aforementioned protected amino acids, and carbodiimides are particularly desirable. Carbodiimides that can be used include DCC, N,N′-diisopropylcarbodiimide, N-ethyl-N′-(3-dimethylaminopropyl)carbodiimide and the like. When activating with these, the protected amino acids can be added directly to the resin together with a racemization suppressor (such as HOBt or HOOBt), or else the protected amino acids can be first activated as symmetrical acid anhydrides or HOBt or HOOBt esters, and then added to the resin.

The solvent used for activating the protected amino acids and condensing with the resin can be selected appropriately from known solvents used in protein synthesis reactions. For example, an acid amide such as N,N-dimethylformamide, N,N-dimethylacetamide or N-methylpyrrolidone, a halogenated hydrocarbon such as methylene chloride or chloroform, an alcohol such as trifluoroethanol, a sulfoxide such as dimethylsulfoxide, an ether such a pyridine, dioxane or tetrahydrofuran, a nitrile such as acetonitrile or propionitrile, an ester such as methyl acetate or ethyl acetate or a suitable mixture of these can be used. The reaction temperature can be selected appropriately from the range known to be suitable for protein bond-forming reactions, and is normally selected from the range of about −20° C. to 50° C. The activated amino acid derivatives are normally used in an excess of 1.5 to 4 times. When a test using a ninhydrin reaction shows condensation to be insufficient, thorough condensation can be achieved by repeating the condensation reaction without removing the protective groups. If sufficient condensation is not obtained even by repeating the reaction, the unreacted amino acids can be acetylated using anhydrous acetic acid or acetylimidazole so as not to affect subsequent reactions.

Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc and the like can be used as protective groups for the amino groups in the raw material.

Carboxyl groups can be protected for example by alkyl esterification (for example, methyl, ethyl, propyl, butyl, t-butyl, cyclopentyl, cyclohexyl, cycloheptyl, cyclooctyl, 2-adamantyl or other straight, branched or cyclic alkyl esterification), aralkyl esterification (for example, benzyl ester, 4-nitrobenzyl ester, 4-methoxybenzyl ester, 4-chlorobenzyl ester or benzhydryl esterification), phenacyl esterification, benzyloxycarbonyl hydrazidation, t-butoxycarbonyl hydrazidation, trityl hydrazidation or the like.

A serine hydroxyl can be protected for example by esterification or etherification. Suitable groups that can be used for such esterification include acetyl and other lower (C₁₋₆) alkanoyl group, benzoyl and other aroyl groups, and benzyloxycarbonyl, ethoxycarbonyl and other groups derived from carbonic acid and the like. Groups suited to etherification include for example benzyl, tetrahydropyranyl and t-butyl groups and the like.

Bzl, Cl₂-Bzl, 2-nitrobenzyl, Br-Z, t-butyl and the like for example can be used as protective groups for the phenolic hydroxyl group of tyrosine.

Tos, 4-methoxy-2,3,6-trimethylbenzenesulfonyl, DNP, benzyloxymethyl, Bum, Boc, Trt, Fmoc and the like for example can be used as protective groups for the imidazole of histidine.

The corresponding acid anhydride, azide, active ester (ester of an alcohol such as pentachlorophenol, 2,4,5-trichlorophenol, 2,4-dinitrophenol, cyanomethyl alcohol, paranitrophenol, HONB, N-hydroxysuccinimide, N-hydroxyphthalimide or HOBt for example) or the like for example can be used as a material in which the carboxyl groups of the raw material have been activated. The corresponding phosphoric acid amide can be used as a material in which the amino groups of the raw material have been activated.

Methods of removing (eliminating) protective groups include for example contact reduction in a flow of hydrogen gas in the presence of a catalyst such as Pd-black or Pd-carbon, acid treatment with anhydrous hydrogen fluoride, methanesulfonic acid, trifluoromethanesulfonic acid, trifluoracetic acid or a mixed liquid of these or the like, base treatment with diisopropyl ethylamine, triethylamine, piperidine, piperazine or the like, or reduction with sodium in liquid ammonia. A elimination reaction by such acid treatment is generally performed at a temperature of about −20° C. to 40° C., and in the case of acid treatment it is useful to add a cation capture agent such as anisole, phenol, thioanisole, metacresol, paracresol, dimethylsulfide, 1,4-butanedithiol, 1,2-ethanedithiol or the like. Moreover, a 2,4-dinitrophenyl group used as the imidazole protective group of histidine is removed by thiophenol treatment, while a formyl group used as an indole protective group of tryptophan can be deprotected by acid treatment in the presence of 1,2-ethanedithiol, 1,4-butanedithiol or the like or removed by alkali treatment using diluted sodium hydroxide solution, diluted ammonia or the like.

Known groups and methods can be selected appropriately for protection and protective groups of functional groups that should not contribute to reaction of the raw material, elimination of such groups, and activation of functional groups contributing to the reaction.

Another method for obtaining an amide of a protein or partial peptide is for example to first amidate and protect the α-carboxy groups of the carboxy terminal amino acids, then elongate the peptide (protein) chains to the desired length on the amino group side to manufacture a protein or partial peptide with only the protective groups of the α-amino groups removed at the N-terminal of the peptide chain and a protein or partial peptide with only the protective groups of the carboxyl groups removed at the C-terminal, and condense these proteins in a mixed solvent as described above. The details of the condensation reaction are similar to those described above. The protected protein or peptide obtained by condensation can then be purified, and all the protective groups can then be removed by the aforementioned methods to obtain the desired crude protein or peptide. This crude protein or peptide can be purified using various known purification techniques, and the principal fraction can then be freeze-dried to obtain an amide of the desired protein or peptide.

To obtain an ester of a protein or peptide, for example the α-carboxyl groups of the carboxy-terminal amino acids can be condensed with an alcohol to obtain an amino acid ester, and an ester of the desired protein or peptide can be obtained in the same way as an amide of the protein or peptide.

A partial peptide or salt thereof used in the present invention can be manufactured by well-known peptide synthesis methods, or by cleaving the protein used in the present invention with a suitable peptidase. The peptide synthesis method can be either a solid-phase synthesis method or liquid-phase synthesis method for example. That is, partial peptides or amino acids capable of forming the partial peptide used in the present invention can be condensed with the residual part, and if the product has protective groups these can be desorbed to manufacture the target peptide. Examples of known condensation methods and protective group elimination methods are described for example in (i) through (v) below.

(i) M. Bodanszky and M. A. Ondetti, Peptide Synthesis, Interscience Publishers, New York (1966)

(ii) Schroeder and Luebke, The Peptide, Academic Press, New York (1965)

(iii) Izumiya, Nobuo et al., Peptide Gosei no Kiso to Jikken, Maruzen (1975)

(iv) Yajima, Haruaki and Sakakibara, Shumpei Seikagaku Jikken Koza 1, Tanpakushitsu no Kagaku IV, 205 (1977)

(v) Yajima, Haruaki Ed., Zoku-Iyakuhin no Kaihatsu, Vol. 14, Peptide Gosei, Hirokawa Shoten

After the reaction, the partial peptide used in the present invention can be purified and isolated by normal purification methods, such as a combination of solvent extraction, distillation, column chromatography, liquid chromatography, recrystallization and the like. When the partial peptide obtained by these methods is in a free form, it can be converted into the corresponding salt by known methods or similar methods, or conversely if it is obtained as a salt, it can be converted into the free form or another salt by known methods or similar methods.

A polynucleotide coding for a protein used in the present invention can be any containing a nucleotide sequence coding for the protein used in the present invention described above. Preferably it is DNA. DNA in this case may be genome DNA, a genome DNA library, cDNA from the aforementioned cells or tissues, a cDNA library from the aforementioned cells or tissue or synthetic DNA.

The vector used for the library may be a bacteriophage, plasmid, cosmid, phagemid or the like. A total RNA or mRNA fraction can also be purified from the aforementioned cells or tissues and amplified by direct Reverse Transcription Polymerase Chain Reaction (hereunder abbreviated as “RT-PCR”).

DNA coding for a protein used in the present invention may be for example (1) DNA containing a nucleotide sequence represented by SEQ ID NO:2, SEQ ID NO:4, SEQ ID NO:6, SEQ ID NO:8, SEQ ID NO:10, SEQ ID NO:12, SEQ ID NO:14 or SEQ ID NO:16 or DNA that contains a nucleotide sequence that hybridizes under highly stringent conditions with a nucleotide sequence represented by SEQ ID NO:2, SEQ ID NO:4, SEQ ID NO:6, SEQ ID NO:8, SEQ ID NO:10, SEQ ID NO:12, SEQ ID NO:14 or SEQ ID NO:16 and that codes for a protein having substantially the same properties as a protein containing an amino acid sequence represented by the aforementioned SEQ ID NO:1, SEQ ID NO:3, SEQ ID NO:5, SEQ ID NO:7, SEQ ID NO:9, SEQ ID NO:11, SEQ ID NO:13 or SEQ ID NO:15, or (2) DNA containing the nucleotide sequence represented by SEQ ID NO:18, or DNA that contains a nucleotide sequence that hybridizes under highly stringent conditions with the nucleotide sequence represented by SEQ ID NO:18, and that codes for a protein having substantially the same properties as a protein containing an amino acid sequence represented by the aforementioned SEQ ID NO:17.

For example, DNA containing a nucleotide sequence having about 50% or greater or preferably about 60% or greater or more preferably about 70% or greater or still more preferably about 80% or greater or especially about 90% or greater or ideally about 95% or greater homology with a nucleotide sequence represented by SEQ ID NO:2, SEQ ID NO:4, SEQ ID NO:6, SEQ ID NO: 8, SEQ ID NO:10, SEQ ID NO:12, SEQ ID NO:14 or SEQ ID NO:16 can be used as the DNA that hybridizes under highly stringent conditions with a nucleotide sequence represented by SEQ ID NO:2, SEQ ID NO:4, SEQ ID NO:6, SEQ ID NO:8, SEQ ID NO:10, SEQ ID NO:12, SEQ ID NO:14 or SEQ ID NO:16.

For example, DNA containing a nucleotide sequence having about 50% or greater or preferably about 60% or greater or more preferably about 70% or greater or still more preferably about 80% or greater or especially about 90% or greater or ideally about 95% or greater homology with the nucleotide sequence represented by SEQ ID NO:18 can be used as the DNA sequence that hybridizes under highly stringent conditions with the nucleotide sequence represented by SEQ ID NO:18.

Nucleotide sequence homology may be calculated using the homology calculation algorithm NCBI BLAST (National Center for Biotechnology Information Basic Local Alignment Search Tool), under the following conditions (expected value=10; gaps allowed; filtering ON; match score=1, mismatch score=−3).

Hybridization can be accomplished by known methods or similar methods, such as for example the methods described in Molecular Cloning 2nd (J. Sambrook et al., Cold Spring Harbor Lab. Press, 1989). When using a commercial library, it can be accomplished according to the methods described in the attached library. More preferably, it is accomplished under highly stringent conditions.

Highly stringent conditions are for example conditions of sodium concentration about 19 to 40 mM or preferably about 19 to 20 mM, and temperature about 50 to 70° C. or preferably about 60 to 65° C. In particular, a sodium concentration of about 19 mM and a temperature of about 65° C. are the most desirable.

More specifically, DNA containing the nucleotide sequence represented by SEQ ID NO:2 can be used as DNA coding for a protein containing the amino acid sequence represented by SEQ ID NO:1, DNA containing the nucleotide sequence represented by SEQ ID NO:4 as DNA coding for a protein containing the amino acid sequence represented by SEQ ID NO:3, DNA containing the nucleotide sequence represented by SEQ ID NO:6 as DNA coding for a protein containing the amino acid sequence represented by SEQ ID NO:5, DNA containing the nucleotide sequence represented by SEQ ID NO:8 as DNA coding for a protein containing the amino acid sequence represented by SEQ ID NO:7, DNA containing the nucleotide sequence represented by SEQ ID NO:10 as DNA coding for a protein containing the amino acid sequence represented by SEQ ID NO:9, DNA containing the nucleotide sequence represented by SEQ ID NO:12 as DNA coding for a protein containing the amino acid sequence represented by SEQ ID NO:11, DNA containing the nucleotide sequence represented by SEQ ID NO:14 as DNA coding for a protein containing the amino acid sequence represented by SEQ ID NO:13, DNA containing the nucleotide sequence represented by SEQ ID NO:16 as DNA coding for a protein containing the amino acid sequence represented by SEQ ID NO:15, and DNA containing the nucleotide sequence represented by SEQ ID NO:18 as DNA coding for a protein containing the amino acid sequence represented by SEQ ID NO:17.

DNA coding for a partial peptide used in the present invention may be any containing a nucleotide sequence coding for a partial peptide used in the present invention as described above. It may be either genome DNA, a genome DNA library, cDNA from the aforementioned cells or tissues, a cDNA library from the aforementioned cells or tissues or synthetic DNA.

DNA including part of DNA containing a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18, or DNA that contains a nucleotide sequence that hybridizes under highly stringent conditions with a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18 and that contains part of DNA coding for a protein having substantially equivalent activity to that of a protein used in the present invention or the like can be used as DNA coding for a partial peptide used in the present invention.

DNA capable of hybridizing with a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18 is defined as above.

Hybridization methods and highly stringent hybridization methods similar to those described above can be used.

DNA coding completely for a protein or partial peptide used in the present invention (which may be simply called a “protein used in the present invention” when explaining the cloning and expression of DNA coding therefor) can be cloned either by PCR amplification using synthetic DNA primers having part of a nucleotide sequence coding for the protein used in the present invention, or by selecting by hybridization DNA incorporated into a suitable vector that has been labeled with a DNA fragment or synthetic DNA coding for some or all regions of the protein used in the present invention. Hybridization can be accomplished for example by the methods described in Molecular Cloning 2nd Edition (J. Sambrook et al., Cold Spring Harbor Lab. Press, 1989). When using a commercial library, it can be accomplished in accordance with the methods described in the attached manual.

A DNA nucleotide sequence can be altered by PCR using a known kit such as Mutan™-super Express Km (Takara), or Mutan™-K (Takara), in accordance with a known method such as ODA-LA PCR, Gapped duplex, Kunkel or the like or a similar method.

According to the objective, DNA coding for a cloned sequence can be used as is or after digestion with a restriction enzyme or addition of a linker as desired. This DNA may have ATG as a translation initiation codon at the 5′ end or TAA, TGA or TAG as a translation termination codon at the 3′ end. This translation initiation codon and translation termination codon can be added using suitable synthetic DNA adapters.

An expression vector for a protein used in the present invention can be manufactured for example by (i) excising a target DNA fragment from DNA coding for the protein used in the present invention, and (ii) linking this DNA fragment downstream from a promoter in a suitable expression vector.

Vectors that can be used include E. coli-derived plasmids (such as pBR322, pBR325, pUC12 or pUC13), B. subtilis-derived plasmids (such as pUB110, pTP5 or pC194), yeast-derived plasmids (such as pSH19 or pSH15), α-phages and other bacteriophages, retroviruses, vaccinia virus, baculoviruses and other animal viruses and the like as well as pA1-11, pXT1, pRc/CMV, pRc/RSV, pcDNAI/Neo and the like.

Any suitable promoter corresponding to the host used to express the gene can be used as a promoter in the present invention. When an animal cell is used as the host, examples include the SRα promoter, SV40 promoter, LTR promoter, CMV promoter, HSV-TK promoter and the like.

Of these, it is desirable to use a CMV (cytomegalovirus) promoter, SRα promoter or the like. When the host is an Escherichia, it is desirable to use a trp promoter, lac promoter, recA promoter, λP_{L} promoter, lpp promoter, T7 promoter or the like, while when the host is a Bacillus it is desirable to use an SPO1 promoter, SPO2 promoter, penP promoter or the like, and when the host is a yeast it is desirable to use PHO5 promoter, PGK promoter, GAP promoter, ADH promoter or the like. When the host is an insect cell, a polyhedrin promoter, P10 promoter or the like is preferred.

In addition to these, enhancers, splicing signals, poly-A addition signals, selection markers, SV40 replication origins (sometimes abbreviated as SV40 ori) and the like may also be included in the vector as desired. Examples of selection signals include the dihydrofolate reductase (hereunder sometimes abbreviated as dhfr) gene [methotrexate (MTX) resistant], ampicillin resistance gene (sometimes abbreviated below as Amp^{r}), neomycin resistance gene (G418 resistant, sometimes abbreviated below as Neo^{r}) and the like. In particular, when using dhfr gene-deficient Chinese hamster cells with the dhfr gene as the selection marker, the target gene can be selected even with medium containing no thymidine.

A signal sequence matching the host can also be added as necessary to the N-terminal end of a protein used in the present invention. When the host is an Escherichia, PhoA signal sequence, OmpA signal sequence or the like can be used, and when the host is a Bacillus, an α-amylase signal sequence, subtilisin signal sequence or the like can be used, while when the host is a yeast, an MFα signal sequence, SUC2 signal sequence or the like can be used, and when the host is an animal cells an insulin signal sequence, α-interferon signal sequence, antibody molecule signal sequence or the like can be used.

A transformant can be produced using a vector constructed in this way that contains DNA coding for a protein used in the present invention.

Hosts that can be used include Escherichia bacteria, Bacillus bacteria, yeasts, insect cells, insects, animal cells and the like.

Specific examples of Escherichia bacteria include Escherichia coli K12-DH1 (Proc. Natl. Acad. Sci. USA, Vol. 60, 160 (1968)), JM103 (Nucleic Acids Research Vol. 9, 309 (1981)), JA221 (Journal of Molecular Biology, Vol. 120, 517 (1978)), HB101 (Journal of Molecular Biology, Vol. 41, 459 (1969)), C600 (Genetics, Vol. 39, 440 (1954)) and the like.

Examples of Bacillus bacteria include Bacillus subtilis MIN 14 (Gene, Vol. 24, 255 (1983)), 207-21 (Journal of Biochemistry, Vol. 95, 87 (1984)) and the like.

Examples of yeasts include Saccharomyces cerevisiae AH22, AH22R⁻, NA87-11A, DKD-5D and 20B-12, Schizosaccharomyces pombe NCYC1913 and NCYC2036, and Pichia pastoris KM71 and the like.

Examples of insect cells include cell strains derived from Spodoptera frugiperda larvae (Sf cells), MG1 cells from the midgut of Trichoplusia ni, High Five™ cells from Trichoplusia ni eggs, cells from Mamestra brassicae or cells from Estigmena acrea and the like when the virus is AcNPV. When the virus is BmNPV, cell strains from silkworms (Bombyx mori N cells; BmN cells) and the like can be used. Examples of such Sf cells include Sf9 cells (ATCC CRL1711), Sf21 cells (Vaughn, J. L. et al., In Vivo, 13, 213-217, (1977)) and the like.

Silkworm larvae or the like can be used as insects (Maeda et al., Nature, Vol. 315, 592 (1985)).

Animal cells that can be used include monkey COS-7 cells, Vero cells, Chinese hamster CHO cells (hereunder abbreviated as CHO cells), dhfr gene-deficient Chinese hamster CHO cells (hereunder abbreviated as CHO (dhfr⁻) cells, mouse L cells, mouse AtT-20, mouse myeloma cells, mouse ATDC5 cells, rat GH3, human FL cells and the like.

Escherichia bacteria can be transformed in accordance with the methods described in Proc. Natl. Acad. Sci. USA, Vol. 69, 2110 (1972) and Gene, Vol 17, 107 (1982) and the like for example.

Bacillus bacteria can be transformed in accordance with the methods described in Molecular 7 General Genetics, Vol. 168, 111 (1979) and the like for example.

Yeasts can be transformed in accordance with the methods described in Methods in Enzymology, Vol. 194, 182-187 (1991) and Proc. Natl. Acad. Sci. USA, Vol. 75, 1929 (1978) and the like for example.

Insect cells or insects can be transformed in accordance with the methods described in Bio/Technology, 6, 47-55 (1988) and the like for example.

Animal cells can be transformed in accordance with the methods described in Saibo Kogaku Bessatsu 8, Shin Saibo Kogaku Jikken Protocol 263-267 (1995) (Shujunsha) and Virology, Vol. 52, 456 (1973) for example.

In this way, a transformant can be obtained that has been transformed with an expression vector containing DNA coding for a protein.

When culturing a transformant for which the host is an Escherichia or Bacillus bacterium, liquid medium is suitable as the medium for culture, with the carbon sources, nitrogen sources, inorganic substance and the like necessary for growth of the transformant included therein. Examples of carbon sources include glucose, dextrin, soluble starch, sucrose and the like for example, while examples of nitrogen sources include ammonium salts, nitrates, corn steep liquor, peptone, casein, meat extract, soybean cake, potato extract and other inorganic and organic materials, and examples of inorganic substance include calcium chloride, sodium dihydrogenphosphate, magnesium chloride and the like. Yeast extract, vitamins, growth promotion factors and the like may also be added. The pH of the medium is preferably about 5 to 8.

The medium for culturing an Escherichia bacterium is preferably M9 medium (Miller, Journal of Experiments in Molecular Genetics, 431-433, Cold Spring Harbor Laboratory, New York, 1972) containing glucose and casamino acids. A chemical such as 30-indolylacrylic acid can also be added as necessary to cause the promoter to act efficiently.

When the host is an Escherichia, culture is normally performed for about 3 to 24 hours at about 15 to 43° C., with aeration and agitation as necessary.

When the host is a Bacillus, culture is normally performed for about 6 to 24 hours at about 30 to 40° C., with aeration and agitation as necessary.

When culturing a transformant for which the host is a yeast, the medium may be for example Burkholder minimal medium (Bostian, K. L. et al., Proc. Natl. Acad. Sci. USA, Vol. 77, 4505 (1980)) or SD medium containing 0.5% casamino acids (Bitter, G. A. et al., Proc. Natl. Acad. Sci. USA, Vol. 81, 5330 (1984)). The pH of the medium is preferably adjusted to about 5 to 8. Culture is normally performed for about 24 to 72 hours at about 20° C. to 35° C., with aeration and agitation as necessary.

When culturing a transformant for which the host is an insect cell or insect, Grace's Insect Medium (Grace, T. C. C., Nature, 195, 788 (1962) with 10% immobilized bovine serum or other additives added thereto as appropriate can be used as the medium. The pH of the medium is preferably adjusted to about 6.2 to 6.4. Culture is normally performed for about 3 to 5 days at about 27° C., with aeration and agitation as necessary.

When culturing a transformant for which the host is an animal cells, the medium may be for example MEM medium containing about 5 to 20% fetal bovine serum (Science, Vol. 122, 501 (1952)), DMEM medium (Virology, Vol. 8, 396 (1959)), RPMI 1640 medium (the Journal of the American Medical Association, Vol. 199, 519 (1967)), 199 medium (Proceeding of the Society for the Biological Medicine, Vol. 73, 1 (1950)) or the like. The pH is preferably adjusted to about 6 to 8. Culture is normally performed for about 15 to 60 hours at about 30° C. to 40° C., with aeration and agitation as necessary.

In this way, a protein used in the present invention can be produced in the cells or cell membranes or outside the cells of a transformant.

The protein used in the present invention can be isolated and purified from such a culture by the following methods for example.

To extract a protein used in the present invention from cultured bacteria or cells, a method can be used in which the bacteria or cells are collected after culture by known methods, suspended in a suitable buffer and disrupted by ultrasound, lisozyme and/or freeze-drying treatment, and a crude protein extract is then obtained by centrifugation or filtration. A protein denaturant such as urea or guanidine hydrochloride or a surfactant such as Triton X-100™ can also be included in the buffer. When the protein is excreted in the culture liquid, after completion of culture the bacteria or cells can be separated by known methods from the supernatant, which is then collected.

The protein contained in culture supernatant or extract thus obtained can be purified by a suitable combination of well-known separation and purification methods. Examples of such known separation and purification methods include salting out, solvent precipitation and other methods using solubility, dialysis, ultrafiltration, gel filtration, SDS-polyacrylamide gel electrophoresis and other methods using primarily differences in molecular weight, ion exchange chromatography and other methods using differences in charge, affinity chromatography and other methods using differences in hydrophobicity, isoelectric point electrophoresis and other methods using differences in isoelectric point and the like.

When the protein obtained in this way is obtained in a free form, it can be converted into a salt by known methods or their equivalents, or conversely when it is obtained as a salt it can be converted by known methods or their equivalents into a free form or another salt.

By applying a suitable protein-modifying enzyme to the protein produced by a recombinant, either before or after purification it is also possible to modify the protein as desired or partially remove polypeptides. Trypsin, chymotrypsin, arginyl endopeptidase, protein kinase, glycosidase or the like can be used as the protein-modifying enzyme.

The presence of a protein used in the present invention produced in this way can be measured by Western blotting or enzyme immunoassay using a specific antibody.

An antibody to a protein used in the present invention, or a partial peptide or salt thereof, may be either a polyclonal antibody or a monoclonal antibody as long as it is an antibody that recognizes the protein used in the present invention or a partial peptide or salt thereof.

An antibody to a protein used in the present invention or a partial peptide or salt thereof (which may be abbreviated simply as “a protein used in the present invention” in the following explanation of antibodies) can be manufactured by known antibody or antiserum manufacturing methods using a protein used in the present invention as the antigen.

### [Preparation of Monoclonal Antibody]

### (a) Preparation of Monoclonal Antibody-Producing Cells

A protein used in the present invention is administered by itself or together with a carrier or diluent to a warm-blooded animal at a site capable of antibody production. Complete Freund's adjuvant or incomplete Freund's adjuvant can also be administered at the time of administration in order to enhance antibody production. The protein is normally administered a total of 2 to 10 times at 2 to 6 week intervals. Examples of warm-blooded animals that can be used include monkeys, rabbits, dogs, guinea pigs, mice, rats, sheep, goats and chickens, but mice and rats are preferred.

When preparing monoclonal antibody-producing cells, individuals with confirmed antibody titer can be selected from warm-blooded animals (such as mice) that have been immunized with the antigen, the spleens or lymph nodes can be extracted 2 to 5 days after the final immunization, and the antibody-producing cells contained in these can be fused to myeloma cells from the same or different animal species to prepare a monoclonal antibody-producing hybridoma. Antibody titer in antiserum can be measured for example by first reacting the labeled protein (described below) with antiserum, and then measuring the activity of a labeling agent bound to the antibody. The fusing operation can be performed by known methods, such as those of Köhler and Milstein (Nature, 256, 495 (1975)). Examples of fusion promoters include polyethylene glycol (PEG) and sendai virus, but PEG is preferred.

Examples of myeloma cells include NS-1, P3U1, SP2/0, AP-1 and other myeloma cells from warm-blooded animals, but P3U1 is used by preference. With the ratio of number of antibody-producing cells (spleen cells) used to myeloma cells in the range of about 1:1 to 20:1, PEG (preferably PEG1000 to PEG 6000) is added at a concentration of about 10 to 80%, and the cells are incubated for 1 to 10 minutes at 20 to 40° C. or preferably 30 to 37° C. to efficiently fuse the cells.

The monoclonal antibody-producing hybridoma can be screened by a variety of methods, such as for example by a method of adding hybridoma culture serum to a solid phase (such as a microplate) having the protein antigen adsorbed thereon directly or together with a carrier, then adding protein A or an anti-immunoglobulin antibody (an anti-mouse immunoglobulin antibody when the cells used for cell fusion are mouse cells) labeled with a radioactive substance or enzyme, and detecting the monoclonal antibody bound to the solid phase, or by a method of adding hybridoma culture serum to a solid phase having an anti-immunoglobulin antibody or protein A bound thereon, adding the protein labeled with a radioactive substance or enzyme, and detecting the monoclonal antibody bound to the solid phase.

Monoclonal antibody selection can be accomplished by known methods or similar methods. It is normally performed in animal cell medium with HAT (hypoxanthine, aminopterin, thymidine) added thereto. The medium for selection and breeding can be may be any medium in which the hybridoma can grow. For example, RMPI 1640 medium containing 1 to 20% or preferably 10 to 20% bovine fetal serum, GIT medium containing 1 to 10% bovine fetal serum (Wako Pure Chemical Industries) or serum-free medium for hybridoma culture (SFM-101, Nissui Pharmaceutical Co.) or the like can be used. The culture temperature is normally 20 to 40° C. or preferably about 37° C. The culture time is normally 5 days to 3 weeks or preferably 1 to 2 weeks. Culture is normally performed in 5% carbon dioxide gas. The antibody titer of the hybridoma culture serum can be measured in the same way as the antibody titer of antiserum as described above.

### (b) Purification of Monoclonal Antibody

The monoclonal antibody can be separated and purified by known methods, such as for example by immunoglobulin separation and purification methods (for example, salting out, alcohol precipitation, isoelectric point precipitation, electrophoresis, adsorption-elimination with an ion-exchange body (such as DEAE), ultracentrifugation, gel filtration, or a specific purification method in which the antibody alone is collected with an antigen-bound solid phase or an active adsorbent such as protein A or protein G, and the bonds are released to obtain the antibody).

### [Preparation of Polyclonal Antibody]

A polyclonal antibody of the present invention can be manufactured by known methods or similar methods. For example, the immune antigen (protein antigen) can be prepared by itself or as a fused body together with a carrier protein, a warm-blooded animal can be immunized by the same methods used for the monoclonal antibody above, matter containing an antibody to the protein used in the present invention can be collected from this immune animal, and the antibody can then be separated and purified.

When the immune antigen used to immunize the warm-blooded animal is fused to a carrier protein, the type of carrier protein and the ratio of carrier and hapten can be any that allow the antibody to be efficiently produced in response to immunization with the hapten crosslinked to the carrier, but for example bovine serum albumin, bovine thyroglobulin, hemocyanin or the like can be coupled at a weight ratio of about 0.1 to 20 or preferably about 1 to 5 per 1 of hapten.

A variety of condensation agents can be used to couple the hapten and carrier protein, and active ester reagents containing glutaraldehyde, carbodiimide, maleimide active ester, thiol groups and dithiopyridine groups and the like are used.

The condensation product is administered by itself or together with a carrier or diluent to a warm-blooded animal at a site capable of antibody production. Complete Freund's adjuvant or incomplete Freund's adjuvant can be administered at the same time to enhance antibody-producing ability. Administration is normally performed a total of about 3 to 10 times at 2 to 6 week intervals.

The polyclonal antibody can be collected from the blood or ascites or the like and preferably from the blood of a warm-blood animal immunized by the methods described above.

Polyclonal antibody titer in antiserum can be measured in the same way as antibody titer in antiserum as described above. The polyclonal antibody can be separated and purified by immunoglobulin separation and purification methods in the same way as the monoclonal antibody described above.

An antisense polynucleotide having a nucleotide sequence or part of a nucleotide sequence complementary or substantially complementary to the nucleotide sequence of a polynucleotide (preferably DNA) (such DNA is sometimes called “DNA used in the present invention” in the following explanation of antisense polynucleotides) coding for a protein used in the present invention or its polypeptide may be any antisense polynucleotide having a nucleotide sequence or part of a nucleotide sequence complementary or substantially complementary to the nucleotide sequence of DNA used in the present invention, but is preferably antisense DNA.

A nucleotide sequence substantially complementary to DNA used in the present invention may for example be a nucleotide sequence having about 70% or greater or preferably about 80% or greater or more preferably about 90% or greater or still more preferably about 95% or greater homology with all or part of a nucleotide sequence complementary to DNA used in the present invention (that is, the complement strand of DNA used in the present invention). Out of the total nucleotide sequence of the complement strand of DNA used in the present invention, desirable examples are (i) an antisense polynucleotide having about 70% or greater or preferably about 80% or greater or more preferably about 90% or greater or still more preferably about 95% or greater homology with the complement strand of a part of the nucleotide sequence (such as a nucleotide sequence near the initiation codon) that codes for the N-terminal part of the protein used in the present invention) when the antisense polynucleotide is designed for inhibiting translation and (ii) an antisense polynucleotide having about 70% or greater or preferably about 80% or greater or more preferably about 90% or greater or still more preferably about 95% or greater homology with the complement strand of the total nucleotide sequence of DNA used in the present invention including an intron when the antisense polynucleotide is designed for RNA decomposition with RNaseH.

Specific examples include an antisense polynucleotide having a nucleotide sequence or part of a nucleotide sequence complementary or substantially complementary to the nucleotide sequence of DNA containing a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18, and preferably for example an antisense polynucleotide having a nucleotide sequence or part of a nucleotide sequence complementary to the nucleotide sequence of DNA containing a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18 (more preferably an antisense polynucleotide having a nucleotide sequence complementary to the nucleotide sequence of DNA containing a nucleotide sequence represented by SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18).

An antisense polynucleotide is normally composed of about 10 to 40 or preferably about 15 to 30 nucleotides.

In order to prevent decomposition by nucleases and other hydrolyzing enzymes, the phosphoric acid residues (phosphates) of each of the nucleotides making up the antisense DNA can be replaced with chemically modified phosphoric acid residues such as phosphothioate, methylphosphonate, phosphorodithionate and the like. The sugar (deoxyribose) of each nucleotide may also be replaced with a 2′-O-methylated or other chemically modified sugar structure, or the bases thereof (pyrimidine, purine) may also be chemically modified, as long as it hybridizes with DNA having the nucleotide sequence represented by SEQ ID NO:2. These antisense polynucleotides can be manufactured using a known DNA synthesizer.

In the present invention, an antisense polynucleotide (nucleic acid) corresponding to a protein gene used in the present invention, and capable of blocking replication or expression of that gene, is designed and synthesized based on the nucleotide sequence data of DNA coding for the cloned or sequenced protein. This antisense polynucleotide can hybridize with RNA of the protein gene used in the present invention, and is capable of either interfering with the synthesis or functioning of that RNA, or of regulating and controlling expression of the protein gene used in the present invention through interaction with protein-associated RNA used in the present invention. A polynucleotide complementary to a selected sequence of protein-associated RNA used in the present invention, and a polynucleotide capable of hybridizing specifically with protein-associated RNA used in the present invention, are useful in vivo and ex vivo for regulating and controlling expression of a protein gene used in the present invention, and are also useful for treating and diagnosing disease. The term “corresponding” means homologous or complementary to a nucleotide, nucleotide sequence or specific nucleic acid sequence including a gene. “Correspondence” between a nucleotide, nucleotide sequence or nucleic acid and a protein normally indicates the amino acid sequence of a protein induced from (governed by) a nucleotide (nucleic acid) sequence or its complement. A 5′-terminal hairpin loop, 5′-terminal 6-base pair repeat, 5′-terminal untranslated region, polypeptide translation initiation codon, protein coding region, ORF translation termination codon, 3′-terminal untranslated region, 3′-terminal palindrome region or 3′-terminal hairpin loop or the like of a protein gene can be selected as a desirable object domain, but any domain within the protein gene can also be used.

In terms of the association between the target nucleic acids and a polynucleotide complementary to at least part of the object domain, when the target nucleic acids are capable of hybridizing with the object domain, the target nucleic acids can be called “antisense” with respect to the polynucleotide of the object domain. Examples of antisense polynucleotides include polynucleotides containing 2-deoxy-D-ribose, polynucleotides containing D-ribose, other types of polynucleotides that are the N-glycosides of purine or pyrimidine bases, other polymers having non-nucleotide frameworks (such as commercial protein nucleic acids and synthetic sequence-specific nucleic acid polymers) or other polymers containing special bonds (but which must contain nucleotides having arrangements that allow nucleotide hairpins or nucleotide attachments such as occur in DNA or RNA). These may be double-stranded DNA, single-stranded DNA, double-stranded RNA, single-stranded RNA or DNA:RNA hybrids, and may be unmodified polynucleotides (or unmodified oligonucleotides) or may have a known modification added thereto, such as for example by addition of a known label, capping or methylation, replacement of 1 or more natural nucleotides with analogs, intramolecular nucleotide modification such as with uncharged bonds (methylphosphonate, phosphotriester, phosphoramidate, carbamate, etc.) or with bonds having charge or sulfur-containing bonds (phosphorothioate, phosphorodithioate, etc.) for example, or with a protein (for example a nuclease, nuclease inhibitor, toxin, antibody, signal peptide, poly-L-lysine, etc.), sugar (monosaccharide or the like for example) or other side-chain group, or with an intercalator compound (acridine, psoralen or the like for example), or with a chelate compound (such as a metal, radioactive metal, boron, oxidizing metal or the like), or with an alkylating agent, or with a modified bond (for example, an α-anomeric nucleic acid or the like). The terms “nucleoside”, “nucleotide” and “nucleic acid” mean not only those having purine and pyrimidine bases, but also include those having other modified heterocyclic bases. These may also include methylated purine and pyrimidine, acylated purine and pyrimidine or other heterocyclic rings. The sugar parts of modified nucleotides and modified nucleotides may also be modified, such as for example by substituting a halogen or aliphatic group for 1 or more hydroxyl groups, or by substituting an ether, amine or other functional group.

An antisense polynucleotide of the present invention is RNA, DNA or a modified nucleic acid (RNA, DNA). Specific examples of modified nucleic acids are degradation-resistant sulfurized and thiophosphate derivatives of nucleic acids, and polynucleoside amides and oligonucleoside amides. The antisense polynucleotide of the present invention can be designed for example as follows. That is, the antisense polynucleotide can be made more stable in cells, the cell permeability of the antisense polynucleotide can be enhanced, affinity for the target sense chain can be improved, or the toxicity of the antisense polynucleotide can be reduced if it is toxic. Many such modifications are reported in Pharm. Tech. Japan, Vol. 8, page 247 or page 395, 1992 and Antisense Research and Applications, CRC Press, 1993 and the like.

The antisense polynucleotide of the present invention may also contain altered or modified sugars, bases and bonds, or may be provided in a special form such as a liposome or microsphere, or may be applied by means of gene therapy, or may be provided in an adducted form. A polycation such as polylysine that serves to neutralize the charge of the phosphate structure, or a lipid that enhances interaction with the cell membrane and increases nucleic acid uptake (such as a phospholipid or cholesterol) or another hydrophobic molecule can be use in this adducted form. Suitable lipids for adduction include cholesterol and its derivatives (for example, cholesteryl chloroformate, cholic acid and the like). These can be attached to the 3′-terminal or 5′-terminal of the nucleic acid, or can be attached via a base, sugar or intramolecular nucleoside bond. Examples of other bases include capping bases arranged specifically at the 3′-terminal or 5′-terminal of the nucleic acid, which are used in block degradation by exonuclease, RNase and other nucleases. Examples of such capping bases include polyethylene glycol, tetraethylene glycol and other glycols as well as other hydroxyl protective groups known in the field, but are not limited to these.

The inhibitory activity of the antisense polynucleotide can be investigated using the transformant of the present invention, an in vivo or ex vivo gene expression system of the present invention, or an in vivo or ex vivo translation system of the protein for use in the present invention.

The preventative/therapeutic agent for cancer and other medicament of the present invention are explained in detail below.

A protein comprising substantially the same amino acid sequence represented by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 is sometimes abbreviated as “protein A used in the present invention”, while a protein comprising substantially the same amino acid sequence represented by SEQ ID NO:17 is sometimes abbreviated as “protein B used in the present invention”.

An enzyme belonging to the acyl-CoA synthase family is also sometimes called “protein A used in the present invention”, while fatty acid synthase is sometimes called “protein B used in the present invention”.

Cancer cell apoptosis can be selectively and strongly induced and cancer cells can be selectively and effectively killed by simultaneously inhibiting the activity or expression of protein A used in the present invention and the activity or expression of protein B used in the present invention. Thus, a drug or the like obtained by combining (i) at least one selected from a substance that inhibits the activity of protein A used in the present invention (for example, acyl-CoA synthase activity) and a substance that inhibits expression of a gene for protein A used in the present invention with (ii) at least one selected from a substance that inhibits the activity of protein B used in the present invention (for example, fatty acid synthase activity) and a substance that inhibits expression of a gene for protein B used in the present invention can be used as a safe drug such as for example as a preventative/therapeutic agent for cancer (such as brain tumor, hypophyseal adenoma, glioma, acoustic neurilemmoma, retinal sarcoma, thyroid cancer, throat cancer, cancer of the larynx, tongue cancer, thymic cancer, mesothelial cancer, breast cancer, lung cancer, non-small-cell lung cancer, small-cell lung cancer, stomach cancer, esophageal cancer, duodenal cancer, large intestinal cancer, colon cancer, rectal cancer, liver cancer, hepatocarcinoma, pancreatic cancer, pancreatic endocrine tumor, bile duct cancer, gallbladder cancer, penis cancer, kidney cancer, renal pelvic cancer, ureter cancer, renal cell cancer, testicular cancer, prostate cancer, bladder cancer, vulvar cancer, uterine cancer, cervical cancer, cancer of the uterine body, uterine sarcoma, trophoblastic disease, vaginal cancer, ovarian cancer, ovarian germ cell tumor, skin cancer, malignant myeloma, mycosis fungoides, basal cell cancer, soft tissue sarcoma, malignant lymphoma, Hodgkin's disease, myelodysplastic syndrome, multiple myeloma, leukemia, acute myelocytic leukemia, chronic myelocytic leukemia, acute lymphocytic leukemia, chronic lymphocytic leukemia, adult T-cell leukemia, chronic myeloproliferative disease, pancreatic endocrine tumors, fibrous histiocytoma, leiomyosarcoma, rhabdomyosarcoma, cancers of unknown primary origin and the like), as a cancer cell apoptosis promoting agent, or as a cancer cell proliferation inhibiting agent.

The (i) at least one selected from a substance that inhibits the activity of protein A used in the present invention (for example, acyl-CoA synthase activity) and a substance that inhibits expression of a gene for protein A used in the present invention and the (ii) at least one selected from a substance that inhibits the activity of protein B used in the present invention (for example, fatty acid synthase activity) and a substance that inhibits expression of a gene for protein B used in the present invention may also be the same substance. Since such a substance, that is, a substance that inhibits (i′) the activity of protein A used in the present invention or expression of a gene for protein A used in the present invention and (ii′) the activity of protein B used in the present invention or expression of a gene for protein B used in the present invention, is also capable of simultaneously inhibiting the activity or expression of protein A used in the invention and the activity or expression of protein B used in the present invention, it can be used favorably as a drug such as a preventative/therapeutic agent for cancer, a cancer cell apoptosis promoting agent, or a cancer cell proliferation inhibiting agent.

Examples of substances that inhibit the activity of protein A used in the present invention include for example (a) compounds or salts thereof that inhibit the activity of protein A used in the present invention, (c) antibodies to protein A used in the present invention, and (f) mutants of protein A used in the present invention that have a dominant negative effect on protein A used in the present invention, or polynucleotides coding therefor and the like.

Since (a) a compound or salt thereof that inhibits the activity of protein A used in the present invention is capable of inhibiting the activity of protein A used in the present invention, it can be used favorably as a substance that inhibits the activity of protein A used in the present invention. A compound or salt thereof that inhibits the activity of protein A used in the present invention is not particularly limited as long as it is a compound or salt thereof capable of inhibiting an activity of protein A used in the present invention (such as acyl-CoA synthase activity), but examples include compounds and salts thereof that bind to protein A used in the present invention to thereby inhibit its activity. Such a compound or salt thereof may be a compound selected for example from the peptides, proteins, nonpeptide compounds, synthetic compounds, fermentation products, cell extracts, plant extracts, animal tissue extracts, plasma and the like for example. This compound may be a novel compound or a known compound. Examples of salts of this compound include physiologically acceptable metal salts, ammonium salts, salts with organic bases, salts with inorganic acids, salts with organic acids, salts with basic or acidic amino acids and the like. Desirable examples of metal salts include sodium salts, potassium salts and other alkali metal salts; calcium salts, magnesium salts, barium salts and other alkali earth metal salts; and aluminum salts and the like. Desirable examples of salts with organic bases include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like. Desirable examples of salts with inorganic acids include salts with hydrochloric acid, hydrobromic acid, nitric acid, sulfuric acid, phosphoric acid and the like. Desirable examples of salts with organic acids include salts with formic acid, acetic acid, trifluoracetic acid, propionic acid, phthalic acid, fumaric acid, oxalic acid, tartaric acid, maleic acid, citric acid, succinic acid, malic acid, methanesulfonic acid, benzoic acid, benzenesulfonic acid, p-toluenesulfonic acid and the like. Desirable examples of salts with basic amino acids include salts with arginine, lysine, ornithine and the like, while desirable examples of salts with acidic amino acids include salts with aspartic acid, glutamic acid and the like.

Of these, a physiologically acceptable salt is preferred. Examples include alkali metal salts (for example, sodium salts, potassium salts and the like), alkali earth metal salts (for example, calcium salts, magnesium salts, barium salts and the like) and other inorganic metal salts and ammonium salts and the like when there is an acidic functional group in the compound and, and salts with hydrobromic acid, nitric acid, sulfuric acid, phosphoric acid and other inorganic acids and acetic acid, phthalic acid, fumaric acid, oxalic acid, tartaric acid, maleic acid, citric acid, succinic acid, methanesulfonic acid, p-toluenesulfonic acid and other organic acids when there is a basic functional group in the compound.

A low-molecular-weight compound is preferred as the compound or salt thereof that inhibits the activity of protein A used in the present invention.

Examples of the compound or salt thereof that inhibits the activity of protein A used in the present invention include Triacsin C, 2-bromopalmitic acid or salts of these, and Triacsin C or its salt is preferred.

This compound or its salt may be formulated and administered by ordinary methods.

Examples of compositions for oral administration include solid or liquid dosage forms, such as specifically tablets (including sugar-coated tablets and film-coated tablets), pills, granules, powders, capsules (including soft capsules), syrups, emulsions, suspensions and the like. These compositions can be manufactured by known methods, and may contain carriers, diluents or excipients commonly used in the pharmaceutical field. For example, lactose, starch, sucrose, magnesium stearate and the like can be used as carriers and excipients in tablets.

Examples of compositions for non-oral administration include injections, suppositories and the like, and injections include intravenous injections, subcutaneous injections, intracutaneous injections, muscular injections, drip injections, intraarticular injections and other formulations. These injections can be prepared by known methods, such as by dissolving, suspending or emulsifying the aforementioned substance in a sterile aqueous or oily liquid commonly used for injections. Physiological saline and isotonic fluid containing glucose or other adjuvants and the like can be used as aqueous liquids for injections, and a suitable solubilizer such as an alcohol (for example ethanol), polyalcohol (for example propylene glycol, polyethylene glycol) or non-ionic surfactant (for example polysorbate 80 or HCO-50 (polyoxyethylene (50 mol) adduct of hydrogenated castor oil)) may also be included. Sesame oil, soybean oil and the like can be used as oily liquids, and benzyl benzoate, benzyl alcohol or the like may be included as a solubilizer. The prepared injection liquid is normally packed in a suitable ampoule. A suppository for rectal administration is prepared by mixing the aforementioned substance with a suitable suppository base.

Since (c) an antibody to protein A used in the present invention and preferably an antibody having the effect of neutralizing (reducing or eliminating) the activity of protein A used in the present invention (neutralizing enzyme) is capable of inhibiting the activity of protein A used in the present invention, it can be used favorably as a substance that inhibits the activity of protein A used in the present invention.

This antibody can be administered by itself or as a suitable drug composition. The drug composition used for such administration contains the antibody or its salt with a physiologically acceptable carrier, diluent or excipient. This composition is provided in a dosage form suited to oral or non-oral (such as intravenous) administration. It is preferably provided as an inhalation.

The compositions described above may also contain other active components to the extent that these do not cause undesirable interactions when compounded with the antibody.

Since (f) a mutant of protein A used in the present invention that has a dominant negative effect on protein A used in the present invention, or a polynucleotide coding therefor, is capable of inhibiting the activity of protein A used in the present invention, it can be used favorably as a substance that inhibits the activity of protein A used in the present invention. In this Description, “a mutant of protein A used in the present invention that has a dominant negative effect on protein A used in the present invention” is a protein that, when expressed, has the effect of inhibiting (eliminating or reducing) the activity of protein A used in the present invention (see Taira, Kazunari Ed., Idenshi to Kino Sogai Jikkenho, Yodosha, p. 26-32, 2001, etc.).

Such a mutant of protein A used in the present invention or a polynucleotide coding therefor can be formulated and administered by ordinary methods.

A substance that inhibits expression of protein A used in the present invention is not limited as long as it inhibits expression of protein A used in the present invention, but may be (i) a substance that inhibits transcription of a gene (DNA) coding for protein A used in the present invention into mRNA coding for protein A used in the present invention or (ii) a substance that inhibits translation of mRNA coding for protein A used in the present invention into protein A used in the present invention. (i) A substance that inhibits transcription of a gene (DNA) coding for protein A used in the present invention into mRNA coding for protein A used in the present invention is not particularly limited as long as it inhibits transcription of a gene (DNA) coding for protein A used in the present invention into mRNA coding for protein A used in the present invention, but examples include substances that inhibit transcription of genes (DNA) coding for protein A used in the present invention into mRNA by binding to factors involved in transcription of genes (DNA) coding for protein A used in the present invention into mRNA. (ii) A substance that inhibits translation of mRNA coding for protein A used in the present invention into protein A used in the present invention is not particularly limited as long as it inhibits translation of mRNA coding for protein A used in the present invention into protein A used in the present invention, but examples include substances that inhibit translation of mRNA coding for protein A used in the present invention into protein A used in the present invention by binding to factors involved in translation of mRNA coding for protein A used in the present invention into protein A used in the present invention. Specific examples of such substances that inhibit expression of protein A used in the present invention include (b) a compound or salt thereof that inhibits expression of protein A used in the present invention, (d) an antisense polynucleotide containing a nucleotide sequence or part of a nucleotide sequence complementary or substantially complementary to the nucleotide sequence of a polynucleotide coding for protein A used in the present invention, (e) double-stranded RNA having an RNAi effect on a polynucleotide coding for protein A used in the present invention (such as siRNA or shRNA for a polynucleotide coding for protein A used in the present invention), and (g) a polynucleotide having ribozyme activity with respect to a polynucleotide coding for protein A used in the present invention.

Since (b) a compound or salt thereof that inhibits expression of protein A used in the present invention is capable of suppressing expression of protein A used in the present invention, it can be used favorably as a substance that inhibits expression of protein A used in the present invention. A compound or salt thereof that inhibits expression of protein A used in the present invention is not particularly limited as long as it inhibits expression of protein A used in the present invention, but may be (i) a compound that inhibits transcription of a gene (DNA) coding for protein A used in the present invention into mRNA coding for protein A used in the present invention or (ii) a compound that inhibits translation of mRNA coding for protein A used in the present invention into protein A used in the present invention. (i) A compound that inhibits transcription of a gene (DNA) coding for protein A used in the present invention into mRNA coding for protein A used in the present invention is not particularly limited as long as it inhibits transcription of a gene (DNA) coding for protein A used in the present invention into mRNA, but examples include compounds that inhibit transcription by binding to factors involved in transcription of genes (DNA) coding for protein A used in the present invention into mRNA. (ii) A compound that inhibits translation of mRNA coding for protein A used in the present invention into protein A used in the present invention is not particularly limited as long as it inhibits translation of mRNA coding for protein A used in the present invention into protein A used in the present invention, but examples include compounds that inhibit translation by binding to factors involved in translation of mRNA coding for protein A used in the present invention into protein A used in the present invention.

A compound or salt thereof that inhibits expression of protein A used in the present invention is preferably a low-molecular-weight compound.

Examples of compounds or salts thereof that inhibit expression of protein A used in the present invention include Triacsin C, 2-bromopalmitic acid or salts of these, and Triacsin C or its salt is preferred.

The aforementioned compound or salt thereof can be formulated and administered by ordinary methods in the same way as the aforementioned compound or salt thereof that inhibits the activity of protein A used in the present invention for example.

Since (d) an antisense polynucleotide containing a nucleotide sequence or part of a nucleotide sequence complementary or substantially complementary to the nucleotide sequence of a polynucleotide coding for protein A used in the present invention is of low toxicity, is capable of suppressing expression of a gene coding for protein A used in the present invention and is capable of suppressing expression of protein A used in the present invention, it can be used favorably as a substance that inhibits expression of protein A used in the present invention. Those described above and the like can be used as such antisense polynucleotides.

This antisense polynucleotide can be formulated and administered by known methods.

Moreover, this antisense polynucleotide can be administered orally or non-orally by ordinary methods to humans or mammals (such as rats, rabbits, sheep, pigs, cows, cats, dogs, monkeys and the like), either alone or after insertion into a suitable vector such as a retrovirus vector, adenovirus vector, adenovirus associated virus vector or the like. This antisense polynucleotide can be formulated as is or together with an adjuvant for promoting ingestion or other physiologically acceptable carriers, and can be administered via a gene gun or hydrogel catheter or other catheter. Alternatively, it can be made into an aerosol and administered locally to the trachea as an inhalant.

The antisense polynucleotide can also be formulated as an injection alone or together with a liposome or other carrier and administered intravenously, subcutaneously, into a joint cavity or into a cancer lesion site in order to improve the pharmacokinetics, extend the half-life and improve the efficiency of cellular uptake.

Since (e) double-stranded RNA having an RNAi effect on a polynucleotide coding for protein A used in the present invention (such as siRNA or shRNA for a polynucleotide coding for protein A used in the present invention) is of low toxicity, is capable of inhibiting translation of a gene coding for protein A used in the present invention and is capable of suppressing expression of protein A used in the present invention, it can be used favorably as a substance that inhibits expression of protein A used in the present invention. Examples of such double-stranded RNA having an RNAi effect on a polynucleotide coding for protein A used in the present invention include double-stranded RNA containing part of RNA coding for a protein used in the present invention (such as siRNA (small (short) interfering RNA) or shRNA (small (short) hairpin RNA for a polynucleotide coding for protein A used in the present invention).

Such double-stranded RNA can be designed and manufactured based on the polynucleotide sequence of the present invention in accordance with known methods (see for example Nature, Vol. 411, p. 494, 2001; Patent Publication No. 2002-516062; US Patent Application Disclosure No. 2002/086356, Description; Nature Genetics, Vol. 24, p. 180-183, 2000; Genesis, Vol. 26, p. 240-244, 2000; Nature, Vol. 407, p. 319-320, 2002; Genes & Deve., Vol. 16, p. 948-958, 2002; Proc. Natl. Acad. Sci. USA, Vol. 99, p. 5515-5520, 2002; Science, Vol. 296, p. 550-553, 2002; Proc. Natl. Acad. Sci. USA, Vol. 99, p. 6047-6052, 2002; Nature Biotechnology, Vol. 20, p. 497-500, 2002; Nature Biotechnology, Vol. 20, p. 500-505, 2002; Nucleic Acids Res., Vol. 30, e46, 2002).

The length of double-stranded RNA having an RNAi effect that is used in the present invention is normally 17 to 30 bases or preferably 19 to 27 bases of more preferably 20 to 22 bases.

This double-stranded RNA can be formulated and administered in the same way as the aforementioned antisense polynucleotide.

Since (g) a polynucleotide having ribozyme activity with respect to a polynucleotide coding for protein A used in the present invention is capable of suppressing expression of protein A used in the present invention, it can be used favorably as a substance that inhibits expression of protein A used in the present invention. Such a ribozyme can be designed and manufactured based on the polynucleotide sequence of the present invention in accordance with known methods (see for example Trends in Molecular Medicine, Vol. 7, p. 221, 2001; FEBS Lett., Vol. 228, p. 228, 1988; FEBS Lett., Vol. 239, p. 285, 1988; Nucl. Acids Res., Vol. 17, p. 7059, 1989; Nature, Vol. 323, p. 349, 1986; Nucl Acids Res., Vol. 19, p. 6751, 1991; Protein Eng., Vol. 3, p. 733, 1990; Nucl Acids Res., Vol. 19, p. 3875, 1991; Nucl. Acids Res., Vol. 19, p. 5125, 1991; Biochem. Biophys. Res. Commun., Vol. 186, p. 1271, 1992). For example, it can be manufactured by linking a known ribozyme to part of RNA coding for protein A used in the present invention. The part of RNA coding for protein A used in the present invention may be a part (RNA fragment) near a cleavage site on RNA of the present invention that can be cleaved by a known ribozyme. This ribozyme may be a large ribozyme such as group I intron ribozyme or M1 RNA (a subunit of RNaseP), or may be a hammerhead, hairpin or other small ribozyme (Protein, Nucleic Acid and Enzyme, Vol. 35, p. 2191, 1990). Regarding hammerhead ribozymes, see for example FEBS Lett., Vol. 228, p. 228, 1988; FEBS Lett., Vol. 239, p. 285, 1988; Protein, Nucleic Acid and Enzyme, Vol. 35, p. 2191, 1990; Nucl. Acids Res., Vol. 17, p. 7059, 1989 and the like. Regarding hairpin ribozymes, see for example Nature, Vol. 323, p. 349, 1986; Nucl. Acids Res., Vol. 19, p. 6751, 1991; Kagaku to Seibutsu, Vol. 30, p. 112, 1992 and the like.

This polynucleotide having ribozyme activity can be prepared and administered in the same way as the aforementioned antisense polynucleotide.

Examples of substances that inhibit the activity of protein B used in the present invention include for example (a) compounds and salts thereof that inhibit the activity of protein B used in the present invention, (c) antibodies to protein B used in the present invention and (f) mutants of protein B used in the present invention that have a dominant negative effect on protein B used in the present invention, or polynucleotides coding therefor and the like.

Since (a) a compound or salt thereof that inhibits the activity of protein B used in the present invention is capable of inhibiting the activity of protein B used in the present invention, it can be used favorably as a substance that inhibits the activity of protein B used in the present invention. A compound or salt thereof that inhibits the activity of protein B used in the present invention is not particularly limited as long as it is a compound or salt thereof capable of inhibiting an activity of protein B used in the present invention (such as fatty acid synthase activity), but examples include compounds and salts thereof that bind to protein B used in the present invention to thereby inhibit its activity. Such a compound or salt thereof may be a compound selected for example from the peptides, proteins, nonpeptide compounds, synthetic compounds, fermentation products, cell extracts, plant extracts, animal tissue extracts, plasma and the like for example. This compound may be a novel compound or a known compound. Examples of salts of this compound include those similar to salts of the compound that inhibits the activity of protein B used in the present invention as described above.

A low-molecular-weight compound is preferred as the compound or salt thereof that inhibits the activity of protein B used in the present invention.

Examples of the compound or salt thereof that inhibits the activity of protein B used in the present invention include Cerulenin, C75 or salts of these, and Cerulenin and C75 or salts of these are preferred.

This compound or its salt may be formulated and administered in the same way as the compound or salt thereof that inhibits the activity of protein A used in the present invention as described above.

Since (c) an antibody to protein B used in the present invention and preferably an antibody (neutralizing antibody) having the effect of neutralizing (reducing or eliminating) the activity of protein B used in the present invention is capable of inhibiting the activity of protein B used in the present invention, it can be used favorably as a substance that inhibits the activity of protein B used in the present invention.

This antibody can be formulated and administered in the same way as the aforementioned antibody to protein A used in the present invention.

Since (f) a mutant of protein B used in the present invention that has a dominant negative effect on protein B used in the present invention, or a polynucleotide coding therefor, is capable of inhibiting the activity of protein B used in the present invention, it can be used favorably as a substance that inhibits the activity of protein B used in the present invention. In this Description, “a mutant of protein B used in the present invention that has a dominant negative effect on protein B used in the present invention” is a protein that, when the polynucleotide coding therefor is expressed, has the effect of inhibiting (eliminating or reducing) the activity of protein B used in the present invention (see Taira, Kazunari Ed., Idenshi no Kino Sogai Jikkenho, Yodosha, p. 26-32, 2001, etc.).

Such a mutant of protein B used in the present invention or a polynucleotide coding therefor can be formulated and administered by ordinary methods.

A substance that inhibits expression of protein B used in the present invention is not limited as long as it inhibits expression of protein B used in the present invention, but may be (i) a substance that inhibits transcription of a gene (DNA) coding for protein B used in the present invention into mRNA coding for protein B used in the present invention or (ii) a substance that inhibits translation of mRNA coding for protein B used in the present invention into protein B used in the present invention. (i) A substance that inhibits transcription of a gene (DNA) coding for protein B used in the present invention into mRNA coding for protein B used in the present invention is not particularly limited as long as it inhibits transcription of a gene (DNA) coding for protein B used in the present invention into mRNA coding for protein B used in the present invention, but examples include substances that inhibit transcription of genes (DNA) coding for protein B used in the present invention into mRNA by binding to factors involved in transcription of genes (DNA) coding for protein B used in the present invention into mRNA. (ii) A substance that inhibits translation of mRNA coding for protein B used in the present invention into protein B used in the present invention is not particularly limited as long as it inhibits translation of mRNA coding for protein B used in the present invention into protein B used in the present invention, but examples include substances that inhibit translation of mRNA coding for protein B used in the present invention into protein B used in the present invention by binding to factors involved in translation of mRNA coding for protein B used in the present invention into protein B used in the present invention. Specific examples of such substances that inhibit expression of protein B used in the present invention include (b) a compound or salt thereof that inhibits expression of protein B used in the present invention, (d) an antisense polynucleotide containing a nucleotide sequence or part of a nucleotide sequence complementary or substantially complementary to the nucleotide sequence of a polynucleotide coding for protein B used in the present invention, (e) double-stranded RNA having an RNAi effect on a polynucleotide coding for protein B used in the present invention (such as siRNA or shRNA for a polynucleotide coding for protein B used in the present invention), and (g) a polynucleotide having ribozyme activity with respect to a polynucleotide coding for protein B used in the present invention.

Since (b) a compound or salt thereof that inhibits expression of protein B used in the present invention is capable of suppressing expression of protein B used in the present invention, it can be used favorably as a substance that inhibits expression of protein B used in the present invention. A compound or salt thereof that inhibits expression of protein B used in the present invention is not particularly limited as long as it inhibits expression of protein B used in the present invention, but may be (i) a compound that inhibits transcription of a gene (DNA) coding for protein B used in the present invention into mRNA coding for protein B used in the present invention or (ii) a compound that inhibits translation of mRNA coding for protein B used in the present invention into protein B used in the present invention. (i) A compound that inhibits transcription of a gene (DNA) coding for protein B used in the present invention into mRNA coding for protein B used in the present invention is not particularly limited as long as it inhibits transcription of a gene (DNA) coding for protein B used in the present invention into mRNA, but examples include compounds that inhibit transcription by binding to factors involved in transcription of genes (DNA) coding for protein B used in the present invention into mRNA. (ii) A compound that inhibits translation of mRNA coding for protein B used in the present invention into protein B used in the present invention is not particularly limited as long as it inhibits translation of mRNA coding for protein B used in the present invention into protein B used in the present invention, but examples include compounds that inhibit translation by binding to factors involved in translation of mRNA coding for protein B used in the present invention into protein B used in the present invention.

A compound or salt thereof that inhibits expression of protein B used in the present invention is preferably a low-molecular-weight compound.

Examples of compounds or salts thereof that inhibit expression of protein B used in the present invention include Cerulenin, C75 or salts of these, and Cerulenin and C75 or their salts are preferred.

This compound or salt thereof can be formulated and administered by ordinary methods in the same way as the aforementioned compound or salt thereof that inhibits the expression of protein A used in the present invention.

Since (d) an antisense polynucleotide containing a nucleotide sequence or part of a nucleotide sequence complementary or substantially complementary to the nucleotide sequence of a polynucleotide coding for protein B used in the present invention is of low toxicity, is capable of suppressing expression of a gene coding for protein B used in the present invention and is capable of suppressing expression of protein B used in the present invention, it can be used favorably as a substance that inhibits expression of protein B used in the present invention. Those described above and the like can be used as such antisense polynucleotides.

This antisense polynucleotide can be formulated and administered in the same way as the aforementioned an antisense polynucleotide containing a nucleotide sequence or part of a nucleotide sequence complementary or substantially complementary to the nucleotide sequence of a polynucleotide coding for protein A used in the present invention.

Since (e) double-stranded RNA having an RNAi effect on a polynucleotide coding for protein B used in the present invention (such as siRNA or shRNA for a polynucleotide coding for protein B used in the present invention) is of low toxicity, is capable of inhibiting translation of a gene coding for protein B used in the present invention and is capable of inhibiting expression of protein B used in the present invention, it can be used favorably as a substance that inhibits expression of protein B used in the present invention. Examples of such double-stranded RNA having an RNAi effect on a polynucleotide coding for protein B used in the present invention include double-stranded RNA containing part of RNA coding for a protein used in the present invention (such as siRNA (small (short) interfering RNA) or shRNA (small (short) hairpin RNA) for a polynucleotide coding for protein B used in the present invention).

Such double-stranded RNA can be designed and manufactured based on a polynucleotide sequence of the present invention using methods similar to those used for double-stranded RNA having an RNAi effect on a polynucleotide coding for protein A used in the present invention.

The length of double-stranded RNA having an RNAi effect that is used in the present invention is normally 17 to 30 bases or preferably 19 to 27 bases of more preferably 20 to 22 bases.

This double-stranded RNA can be formulated and administered in the same way as the aforementioned antisense polynucleotide.

Since (g) a polynucleotide having ribozyme activity with respect to a polynucleotide coding for protein B used in the present invention is capable of inhibiting expression of protein B used in the present invention, it can be used favorably as a substance that inhibits expression of protein B used in the present invention. Such a ribozyme can be designed and manufactured based on a polynucleotide sequence of the present invention in accordance with known methods (see for example Trends in Molecular Medicine, Vol. 7, p. 221, 2001; FEBS Lett., Vol. 228, p. 228, 1988; FEBS Lett., Vol. 239, p. 285, 1988; Nucl. Acids Res., Vol. 17, p. 7059, 1989; Nature, Vol. 323, p. 349, 1986; Nucl Acids Res., Vol. 19, p. 6751, 1991; Protein Eng., Vol. 3, p. 733, 1990; Nucl Acids Res., Vol. 19, p. 3875, 1991; Nucl. Acids Res., Vol. 19, p. 5125, 1991; Biochem. Biophys. Res. Commun., Vol. 186, p. 1271, 1992). For example, it can be manufactured by linking a known ribozyme to part of RNA coding for protein B used in the present invention. The part of RNA coding for protein B used in the present invention may be a part (RNA fragment) near a cleavage site on RNA of the present invention that can be cleaved by a known ribozyme. This ribozyme may be a large ribozyme such as group I intron ribozyme or M1 RNA (a subunit of RNaseP), or may be a hammerhead, hairpin or other small ribozyme (Protein, Nucleic Acid and Enzyme, Vol. 35, p. 2191, 1990). See the references described above with respect to hammerhead ribozymes and hairpin ribozymes.

This polynucleotide having ribozyme activity can be prepared and administered in the same way as the aforementioned antisense polynucleotide.

A preventative/therapeutic agent for cancer, cancer cell apoptosis promoting agent or cancer cell proliferation suppressing agent or the like prepared by combining a compound or salt thereof that inhibits the activity of protein A used in the present invention with a compound or salt thereof that inhibit the activity of protein B used in the present invention is preferred as the aforementioned preventative/therapeutic agent for cancer or other medicament of the present invention.

Moreover, since (i) a substance that inhibits the activities of an enzyme belonging to the acyl-CoA synthase family and fatty acid synthase and (ii) a substance that inhibits expression of a gene for an enzyme belonging to the acyl-CoA synthase family and expression of a fatty acid synthase gene and the like are capable of simultaneously inhibiting the activity or expression of protein A used in the present invention and inhibiting the activity or expression of protein B used in the present invention, they can be used favorably as the aforementioned preventative/therapeutic agent for cancer or other medicament of the present invention.

Moreover, a (i) a substance that inhibits the activities of an enzyme belonging to the acyl-CoA synthase family and fatty acid synthase and (ii) a substance that inhibits expression of a gene for an enzyme belonging to the acyl-CoA synthase family and expression of a fatty acid synthase gene may also be the same substance.

The preventative/therapeutic agent for cancer or other medicament of the present invention may be in a form capable of simultaneously inhibiting the activity or expression of protein A used in the present invention and the activity or expression of protein B used in the present invention. For example, (i) at least one selected from a substance that inhibits the activity of protein A used in the present invention and a substance that inhibits expression of a gene for protein A used in the present invention, and (ii) at least one selected from a substance that inhibits the activity of protein B used in the present invention and a substance that inhibits expression of a gene for protein B used in the present invention can be formulated as a combination preparation in a single drug composition (such as a tablet (including sugar-coated tablets and film-coated tablets), pill, granules, powder, capsule (including soft capsules), syrup, emulsion, suspension, injection, suppository or the like). Moreover, the preventative/therapeutic agent for cancer or other medicament of the present invention may also be a kit comprising (i) a medicament comprising at least one selected from a substance that inhibits the activity of protein A used in the present invention and a substance that inhibits expression of a gene for protein A used in the present invention and (ii) a medicament comprising at least one selected from a substance that inhibits the activity of protein B used in the present invention and a substance that inhibits expression of a gene for protein B used in the present invention. In this case, the (i) medicament comprising at least one selected from a substance that inhibits the activity of protein A used in the present invention and a substance that inhibits expression of a gene for protein A used in the present invention and the (ii) medicament comprising at least one selected from a substance that inhibits the activity of protein B used in the present invention and a substance that inhibits expression of a gene for protein B used in the present invention may be administered with a time interval in between as long as the activity or expression of protein A used in the present invention and the activity or expression of protein B used in the present invention can be simultaneously inhibited, but preferably they are administered simultaneously.

The dosage ratio (or compounding ratio) of the (i) at least one selected from a substance that inhibits the activity of protein A used in the present invention and a substance that inhibits expression of a gene for protein A used in the present invention and the (ii) at least one selected from a substance that inhibits the activity of protein B used in the present invention and a substance that inhibits expression of a gene for protein B used in the present invention in the preventative/therapeutic agent for cancer or other medicament of the present invention differs according to the types and/or combination of substance that inhibits the activity of protein A used in the present invention, substance that inhibits expression of a gene for protein A used in the present invention, substance that inhibits the activity of protein B used in the present invention and substance that inhibits expression of a gene for protein B used in the present invention, and according to the subject of administration, target illness, symptoms, administration route and the like, but for example the weight ratio is about 1:500 to 500:1 or preferably about 1:100 to 100:1 or more preferably 1:10 to 10:1 or still more preferably 1:5 to 5:1.

When using (i) at least one selected from a substance that inhibits the activity of protein A used in the present invention and a substance that inhibits expression of a gene for protein A used in the present invention, and (ii) at least one selected from a substance that inhibits the activity of protein B used in the present invention and a substance that inhibits expression of a gene for protein B used in the present invention as the aforementioned agent, they can be formulated by ordinary means in accordance with the methods described above for example.

For example, in the case of a composition for oral administration examples include solid and liquid dosage forms, such as specifically tablets (including sugar-coated tablets and film-coated tablets), pills, granules, powders, capsules (including soft capsules), syrups, emulsions, suspensions and the like. These compositions can be manufactured by known methods, and may contain carriers, diluents or excipients commonly used in the pharmaceutical field. For example, lactose, starch, sucrose, magnesium stearate and the like can be used as carriers and excipients in tablets.

Examples of compositions for non-oral administration include injections, suppositories and the like, and injections include intravenous injections, subcutaneous injections, intracutaneous injections, muscular injections, drip injections, intraarticular injections and other formulations. Injections can be prepared by known methods, such as by dissolving, suspending or emulsifying the aforementioned substance in a sterile aqueous or oily liquid commonly used for injections. Physiological saline and isotonic fluid containing glucose or other adjuvants and the like can be used as aqueous liquids for injections, and a suitable solubilizer such as an alcohol (for example ethanol), polyalcohol (for example propylene glycol, polyethylene glycol) or non-ionic surfactant (for example polysorbate 80 or HCO-50 (polyoxyethylene (50 mol) adduct of hydrogenated castor oil)) may also be included. Sesame oil, soybean oil and the like can be used as oily liquids, and benzyl benzoate, benzyl alcohol or the like may be included as a solubilizer. The prepared injection liquid is normally packed in a suitable ampoule. A suppository for rectal administration is prepared by mixing the aforementioned substance with a suitable suppository base.

It is convenient to prepare the aforementioned drug composition for oral or non-oral use in a unit dosage form for administration according to the dosages of the active ingredients. Examples of such unit dosage forms for administration include tablets, pills, capsules, injections (ampoules), suppositories and the like, and each dosage form for administration normally contains 5 to 500 mg or particularly 5 to 100 mg in the case of an injection or 10 to 250 mg in the case of other dosage forms of both (i) at least one selected from a substance that inhibits the activity of protein A used in the present invention and a substance that inhibits expression of a gene for protein A used in the present invention, and (ii) at least one selected from a substance that inhibits the activity of protein B used in the present invention and a substance that inhibits expression of a gene for protein B used in the present invention.

Each of the compositions described above may also contain other active components to the extent that these do not produce undesirable side-effects when compounded with the aforementioned substances.

Because a preparation obtained in this way is safe and of low toxicity, it can be administered orally or non-orally to humans or warm-blooded mammals (such as mice, rats, rabbits, sheep, pigs, cows, horses, birds, cats, dogs, monkeys, chimpanzees and the like).

The dosages of these compounds or salts thereof differ according to the effects, the target illness, the subject of administration and the symptoms, administration route and the like, but generally about 0.1 to 100 mg or preferably about 1.0 to 50 mg or more preferably about 1.0 to 20 mg per day of each substance is administered to an adult (body weight 60 kg) in the case of oral administration of (i) at least one selected from a substance that inhibits the activity of protein A used in the present invention and a substance that inhibits expression of a gene for protein A used in the present invention, and (ii) at least one selected from a substance that inhibits the activity of protein B used in the present invention and a substance that inhibits expression of a gene for protein B used in the present invention for the treatment of lung cancer. In the case of non-oral administration, the dosages differ depending on the target illness, the subject of administration, the symptoms, the administration route and the like, but in general it is convenient to administer about 0.01 to 30 mg or preferably about 0.1 to 20 mg or more preferably about 0.1 to 10 mg per day of each substance intravenously to an adult (body weight 60 kg) when administering (i) at least one selected from a substance that inhibits the activity of protein A used in the present invention and a substance that inhibits expression of a gene for protein A used in the present invention, and (ii) at least one selected from a substance that inhibits the activity of protein B used in the present invention and a substance that inhibits expression of a gene for protein B used in the present invention in injectable form for the treatment of lung cancer. In the case of other animals, an equivalent dosage per 60 kg of body weight can be administered.

This preventative/therapeutic agent for cancer or other medicament of the present invention can also be used in combination with hormone therapy agents, anticancer agents (such as a chemotherapy agents, immunotherapeutic agents and drugs that inhibit the activity of cell growth factors and cell growth factor receptors) or the like (hereunder called combined drugs). In this case the administration period is not limited, and these may be administered to the subject of administration either simultaneously or with a time interval in between. The dosages can be selected appropriately based on dosages in clinical use. The compounded ratio of the preventative/therapeutic agent for cancer or other medicament of the present invention and the combined drug can be selected appropriately according to the subject of administration, administration route, target illness, symptoms, combination and the like.

Examples of “hormone therapy agents” include fosfestrol, diethylstilbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, tadenan, mepartricin, raloxifene, ormeloxifene, levormeloxifene, anti-estrogens (e.g., tamoxifen citrate, toremifene citrate and the like), ER down-regulators (e.g., fulvestrant and the like), human chorionic gonadotropin, follicle-stimulating hormone, birth-control pills, mepitiostane, testrolactone, aminoglutethiimide, LH-RH agonists (e.g., goserelin acetate, buserelin, leuprorelin and the like), droloxifene, epitiostanol, ethinylestradiol sulfonate, aromatase inhibitors (e.g., fadrozole hydrochloride, anastrozole, retrozole, exemestane, vorozole, formestane and the like), anti-androgens (e.g., flutamide, bicartamide, nilutamide and the like), 5α-reductase inhibitors (e.g., finasteride, dutasteride, epristeride and the like), adrenocorticohormone drugs (e.g., dexamethasone, prednisolone, betamethasone, triamcinolone and the like), androgen synthesis inhibitors (e.g., abiraterone and the like), retinoids and drugs that retard retinoid metabolism (e.g., liarozole and the like), etc. LH-RH agonists (e.g., goserelin acetate, buserelin, leuprorelin and the like) are preferable.

Examples of “chemotherapy agents” include alkylating agents, antimetabolites, anticancer antibiotics, plant-derived anticancer agents and the like.

Examples of “alkylating agents” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucid, carboplatin, cisplatin, miboplatin, nedaplatin, oxaliplatin, altretamine, ambamustine, dibrospidiuim hydrochloride, fotemustine, prednimustine, pumitepa, ribomustin, temozolomide, treosulfan, trophosphamide, zinostatin stimalamer, adozelesin, cystemustine, bizelesin and the like.

Examples of “antimetabolites” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine and the like.

Examples of “anticancer antibiotics” include actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride and the like.

Examples of “plant-derived anticancer agents” include etoposide, etoposide phosphate, vinblastine sulfate, vincristine sulfate, vindesine sulfate, teniposide, paclitaxel, docetaxel, vinorelbine and the like.

Examples of “immunotherapeutic agents (BRM)” include picibanil, krestin, sizofuran, lentinan, ubenimex, interferons, interleukins, macrophage colony-stimulating factor, granulocyte colony-stimulating-factor, erythropoietin, lymphotoxin, BCG vaccine, Corynebacterium parvum, levamisole, polysaccharide K, procodazole and the like.

The “cell growth factors” in the “drugs that inhibit the activity of cell growth factors and cell growth factor receptors” may be any substances that promote cell proliferation, and are normally peptides having a molecular weight of not more than 20,000 that are capable of exhibiting their activity at low concentrations by binding to a receptor, including (1) EGF (epidermal growth factor) or substances possessing substantially the same activity as EGF [e.g., EGF, heregulin (HER2 ligand) and the like], (2) insulin or substances possessing substantially the same activity as insulin [e.g., insulin, IGF (insulin-like growth factor)-1, IGF-2 and the like], (3) FGF (fibroblast growth factor) or substances possessing substantially the same activity as FGF [e.g., acidic FGF, basic FGF, KGF (keratinocyte growth factor), FGF-10 and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), and the like], and the like.

“Cell growth factor receptors” may be any receptors capable of binding to the aforementioned cell growth factors, and specific examples include EGF receptor, heregulin receptor (HER2), insulin receptor, IGF receptor, FGF receptor-1 or FGF receptor-2 and the like.

Examples of “drugs that inhibit the activity of cell growth factors” include trastuzumab (trade mark: Herceptin (anti-HER2 antibody)), imatinib mesilate, ZD1839 or cetuximab, antibodies to VEGF (such as bevacizumab), antibodies to VEGF receptors, gefitinib, erlotinib and the like.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan and the like), topoisomerase II inhibitors (e.g., sobuzoxane and the like), differentiation inducers (e.g., retinoid, vitamin D and the like), angiogenesis inhibitors (e.g., thalidomide, SU11248 and the like), α-blockers (e.g., tamsulosin hydrochloride, naftopidil, urapidil, alfuzosin, terazosin, prazosin, silodosin and the like), serine/threonine kinase inhibitors, endothelin receptor antagonists (e.g., atrosentan and the like), proteosome inhibitors (e.g., bortezomib and the like), Hsp90 inhibitors (e.g., 17-AAG and the like), spirolactone, minoxidil, 11α-hydroxyprogesteron, bone absorption inhibitors and metastasis suppressors (e.g., zoledronic acid, alendronic acid, pamidronic acid, etidronic acid, ibandronic acid, clodronic acid) and the like can also be used.

When bases and amino acids are represented by abbreviations in this Description and in the sequence tables, these are based on the abbreviations given by the IUPAC-IUB Commission on Biochemical Nomenclature, or on abbreviations commonly used in the field. Examples are given below. When an amino acid may have an optical isomer, the L-form is indicated unless otherwise specified.

DNA: Deoxyribonucleic acid

cDNA: Complementary deoxyribonucleic acid

A: Adenine

T: Thymine

G: Guanine

C: Cytosine

RNA: Ribonucleic acid

mRNA: Messenger ribonucleic acid

dATP: Deoxyadenosine triphosphate

dTTP: Deoxythymidine triphosphate

dGTP: Deoxyguanosine triphosphate

dCTP: Deoxycytidine triphosphate

ATP: Adenosine triphosphate

EDTA: Ethylenediaminetetraacetic acid

SDS: Sodium dodecyl sulfate

Gly: Glycine

Ala: Alanine

Val: Valine

Leu: Leucine

Ile: Isoleucine

Ser: Serine

Thr: Threonine

Cys: Cysteine

Met: Methionine

Glu: Glutamic acid

Asp: Aspartic acid

Lys: Lysine

Arg: Arginine

His: Histidine

Phe: Phenylalanine

Tyr: Tyrosine

Trp: Tryptophan

Pro: Proline

Asn: Asparagine

Gln: Glutamine

pGlu: Pyroglutamic acid

Sec: Selenocysteine

Substituents, protective groups and reagents frequently mentioned in this description are abbreviated as follows.

Me: Methyl group

Et: Ethyl group

Bu: Butyl group

Ph: Phenyl group

TC: Thiazolidine-4(R)-carboxamide group

Tos: p-toluenesulfonyl

CHO: Formyl

Bzl: Benzyl

Cl₂-Bzl: 2,6-dichlorobenzyl

Bom: Benzyloxymethyl

Z: Benzyloxycarbonyl

Cl-Z: 2-chlorobenzyloxycarbonyl

Br-Z: 2-bromobenzyloxycarbonyl

Boc: t-butoxycarbonyl

DNP: Dinitrophenyl

Trt: Trityl

Bum: t-butoxymethyl

Fmoc: N-9-fluorenylmethoxycarbonyl

HOBt: 1-hydroxybenzotriazole

HOOBt: 3,4-dihydro-3-hydroxy-4-oxo-1,2,3-benzotriazine

HONB: 1-hydroxy-5-norbornene-2,3-dicarboxyimide

DCC: N,N′-dicyclohexylcarbodiimide

The sequence ID numbers in the sequence tables of this Description represent the following sequences.

### [SEQ ID NO:1]

Represents the amino acid sequence of human AC SI.

### [SEQ ID NO:2]

Represents the nucleotide sequence of cDNA coding for human ACS1.

### [SEQ ID NO:3]

Represents the amino acid sequence of human ACS3

### [SEQ ID NO:4]

Represents the nucleotide sequence of cDNA coding for human ACS3.

### [SEQ ID NO:5]

Represents the amino acid sequence of isoform 1 of human ACS4.

### [SEQ ID NO:6]

Represents the nucleotide sequence of cDNA coding for isoform 1 of human ACS4.

### [SEQ ID NO:7]

Represents the amino acid sequence of isoform 2 of human ACS4.

### [SEQ ID NO:8]

Represents the nucleotide sequence of cDNA coding for isoform 2 of human ACS4.

### [SEQ ID NO:9]

Represents the amino acid sequence of isoform a of human ACS5.

### [SEQ ID NO:10]

Represents the nucleotide sequence of cDNA coding for isoform a of human ACS5.

### [SEQ ID NO:11]

Represents the amino acid sequence of isoform b of human ACS5.

### [SEQ ID NO:12]

Represents the nucleotide sequence of cDNA coding for isoform b of human ACS5.

### [SEQ ID NO:13]

Represents the amino acid sequence of isoform a of human ACS6.

### [SEQ ID NO:14]

Represents the nucleotide sequence of cDNA coding for isoform a of human ACS6.

### [SEQ ID NO:15]

Represents the amino acid sequence of isoform b of human ACS6.

### [SEQ ID NO:16]

Represents the nucleotide sequence of cDNA coding for isoform b of human ACS6.

### [SEQ ID NO:17]

Represents the amino acid sequence of human FAS (GeneBank NP_{—}004095).

### [SEQ ID NO:18]

Represents the nucleotide sequence of cDNA coding for human FAS (GeneBank NM_{—}004104).

### [SEQ ID NO:19]

Represents the nucleotide sequence of a primer used in Example 1.

### [SEQ ID NO:20]

Represents the nucleotide sequence of a primer used in Example 1.

### EXAMPLES

The present invention is explained in more detail below using examples, but the present invention is not limited thereby.

### Example 1

### Acquisition of FAS Inhibitor Resistance by ACS Expression

### (1) Cloning of Human ACS5 and Construction of the Expression Vector

The human ACS5 gene was cloned by PCR from human large intestinal cancer HCT-15 cell cDNA. The following sequences were used as primers for cloning human ACS5.

(SEQ ID NO:19)

5′-AAAGAATTCTATGCTTTTTATCTTTAACTTTTTGTTTTCCC-3′

(SEQ ID NO:20)

5′-AAAGGATCCATAATCCTGGATGTGCTCATACAGGC-3′

(The primers were designed with TAT substituted for the stop codon TAG so that a flag tag could be attached to the 3′-terminal.) The reaction was performed in 35 cycles of 30 second at 94° C., 30 seconds at 65° C. and 4 minutes at 72° C., using AmpliTaq DNA polymerase (Applied Biosystems). Both ends of the resulting cDNA fragment were cleaved with EcoRI and BamHI (Takara Bio) and cloned to pFLAG-CMV5 to obtain pFLAG-CMV-ACS5, and the DNA sequence was confirmed. The resulting cDNA sequence was identical with that of known human ACS5 (AB033899). The resulting ACS5 cDNA was excised from pFLAG-CMV-ACS5 with the FLAG tag attached to the 3′ end, and incorporated into the retrovirus vector pHa-IRES-DHFR (Kage, K. et al., Int. J. Cancer, Vol. 97, p. 626 to 630, 2002) to construct pHa-ACS5-FLAG-IRES-DHFR.

### (2) Establishment of Cells Stably Expressing ACS5

Cells stably expressing ACS5 were established as follows. The retrovirus vector pHa-ACS5-FLAG-IRES-DHFR constructed above and a blank pHa-IRES-DHFR vector were introduced into mouse PA317 fibroblast cells using a mammalian transfection kit (Stratagene), and virus liquid was obtained. Human glioma SF268 cells (ATCC) were then infected with the resulting virus suspension, and selected with 100 ng/ml of methotrexate as a selection agent to obtain the stable expression line SF268/ACS5 and SF268/mock as a control. The cell extracts of each cell line were analyzed by Western blotting using anti-FLAG-M2 antibody (Sigma) and anti-tubulin antibody (Sigma) for the control, and stable expression of the ACS5 protein was confirmed in SF268/ACS5.

### (3) Investigation of Changes in FAS Inhibitor Sensitivity in Cancer Cells Due to ACS Expression

This study was performed using the SF268/ACS5 cell with stable high expression of ACS5 established by the methods above, together with SF268/mock cells as control cells and RPMI1640-10% FBS (fetal bovine serum)-20 mM HEPES (pH 6.5) as the cell culture liquid. The SF268/mock cells and SF268/ACS5 cells were seeded 100,000 cells/well on 6-well plates, and cultured at 37° C. for 7 days with RPMI1640-10% FBS-20 mM HEPES (pH 6.5). Then, these were either left untreated or treated for 24 hours by addition of either 15 μg/ml of Cerulenin ([2R,3S,E,E)-2,3-epoxy-4-oxo-7,10-dodecadienamide] (Sigma)) or 15 μm/ml of C75 ([4-methylene-2-octyl-5-oxotetrahydrofuran-3-carboxylic acid] (Alexis Biochemicals)) as a FAS inhibitor to the medium, after which the number of surviving cells was measured by trypan blue exclusion. The number of surviving cells is shown as a percentage given 100% as the number of untreated SF268/mock cells.

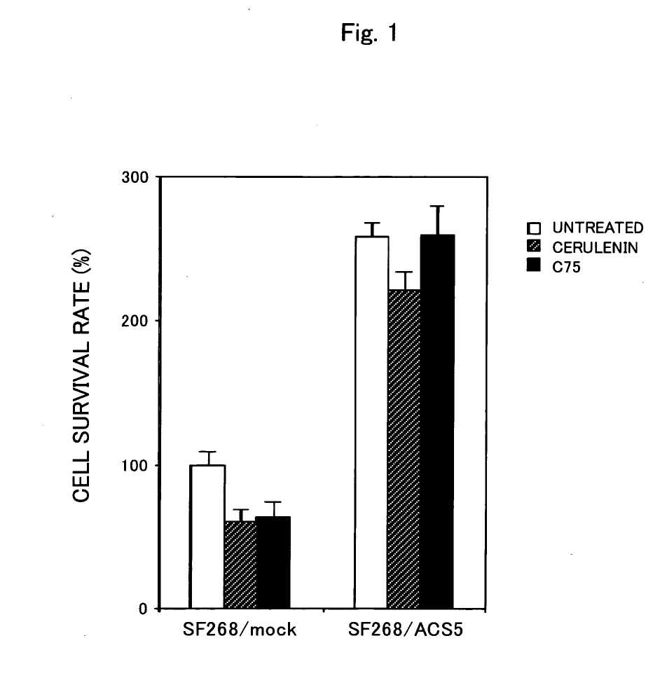

The results are shown in FIG. 1.

Without treatment, the SF268/ACS5 cells with stable high ACS5 expression showed significantly higher cell survival rate than the control SF268/mock cells. Comparing sensitivity to FAS inhibitors, there was a clear growth suppression effect from Cerulenin and C75 in the control SF268/mock cells, but no growth suppression effect from Cerulenin or C75 in the SF268/ACS5 cells with stable high ACS5 expression. This shows that cancer cells overexpressing ACS acquire resistance to FAS inhibitors.

### Example 2

### Investigation of Combined Effect of ACS Inhibitor and FAS Inhibitors

The anti-tumor effects of an ACS inhibitor (Triacsin C [2,4,7-undecatrienal nitrosohydrazone]) and FAS inhibitor (Cerulenin or C75) used in combination was investigated using the lung cancer cell strain NCI-H23 (ATCC). NCI-H23 cells were cultured in RPMI1640-10% FBS. The added drugs Triacsin C, Cerulenin and C75 were all purchased from Sigma. The NCI-H23 cells were seeded on 96-well plates at a concentration of 2000 cells/well, 1 day later (A) Triacsin C, (B) Cerulenin, (C) C75, (D) Triacsin C and Cerulenin and (E) Triacsin C and C75 were added and the cells were treated for 3 days, after which the cell survival rate was measured by the Sulforhodamine B method (Skehan, P. et al., J. Natl. Cancer Inst., Vol. 82, p. 1107 to 1112, 1990, and the results were compared to the results without treatment. The drugs were added at a concentration of 0.3 μM for Triacsin C, 2 μM for Cerulenin and 2 μM for C75. Each test was performed with n=3. As a result, the cell survival rates under drug treatment conditions (A) through (E) above were (A) 56.3±0.5%, (B) 70.2±7.9%, (C) 61.2±3.6%, (D) 13.4±1.4% and (E) 15.3±1.0% given 100% as the cell survival rate without drug treatment. These results show that anti-tumor effects are enhanced by combined use of an ACS inhibitor (Triacsin C) together with a FAS inhibitor (Cerulenin or C75).

### INDUSTRIAL APPLICABILITY

By simultaneously inhibiting the activity or expression of an enzyme belonging to the acyl-CoA synthase (ACS) family and the activity or expression of fatty acid synthase (FAS), it is possible to selectively and strongly induce apoptosis of cancer cells and selectively and effectively kill cancer cells. Thus, 1) a medicament prepared by combining (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family, with (ii) at least one selected from (a) a compound or salt thereof that inhibits the activity of fatty acid synthase, (b) a compound or salt thereof that inhibit expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase, and 2) a medicament containing (i) a substance that inhibits the activities of an enzyme in the acyl-CoA synthase family and of fatty acid synthase and/or (ii) a substance that inhibits expression of a gene belonging to the acyl-CoA synthase family and fatty acid synthase can be used as a safe drug such as for example as a preventative/therapeutic agent for cancer (such as brain tumor, hypophyseal adenoma, glioma, acoustic neurilemmoma, retinal sarcoma, thyroid cancer, throat cancer, cancer of the larynx, tongue cancer, thymic cancer, mesothelial cancer, breast cancer, lung cancer, non-small-cell lung cancer, small-cell lung cancer, stomach cancer, esophageal cancer, duodenal cancer, large intestinal cancer, colon cancer, rectal cancer, liver cancer, hepatocarcinoma, pancreatic cancer, pancreatic endocrine tumor, bile duct cancer, gallbladder cancer, penis cancer, kidney cancer, renal pelvic cancer, ureter cancer, renal cell cancer, testicular cancer, prostate cancer, bladder cancer, vulvar cancer, uterine cancer, cervical cancer, cancer of the uterine body, uterine sarcoma, trophoblastic disease, vaginal cancer, ovarian cancer, ovarian germ cell tumor, skin cancer, malignant myeloma, mycosis fungoides, basal cell cancer, soft tissue sarcoma, malignant lymphoma, Hodgkin's disease, myelodysplastic syndrome, multiple myeloma, leukemia, acute myelocytic leukemia, chronic myelocytic leukemia, acute lymphocytic leukemia, chronic lymphocytic leukemia, adult T-cell leukemia, chronic myeloproliferative disease, pancreatic endocrine tumors, fibrous histiocytoma, leiomyosarcoma, rhabdomyosarcoma, cancers of unknown primary origin and the like), as a cancer cell apoptosis promoting agent, or as a cancer cell proliferation inhibiting agent or the like.

The (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family and (ii) at least one selected from (a) a compound or salt thereof that inhibits the activity of fatty acid synthase, (b) a compound or salt thereof that inhibit expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase may also be the same substance.

The (i) substance that inhibits the activities of an enzyme in the acyl-CoA synthase family and fatty acid synthase and the (ii) substance that inhibits expression of a gene belonging to the acyl-CoA synthase family and fatty acid synthase may also be the same substance.

1. A preventive/therapeutic agent for cancer prepared by combining (i) at least one selected from a substance that inhibits the activity of an enzyme belonging to the acyl-CoA synthase family and a substance that inhibits expression of a gene for an enzyme belonging to the acyl-CoA synthase family, with (ii) at least one selected from a substance that inhibits the activity of fatty acid synthase and a substance that inhibits expression of a fatty acid synthase gene.

2. The preventive/therapeutic agent according to claim 1, wherein the enzyme belonging to the acyl-CoA synthase family is at least one selected from the proteins comprising the same or substantially the same amino acid sequences represented by SEQ ID NO:1, SEQ ID NO:3, SEQ ID NO:5, SEQ ID NO:7, SEQ ID NO:9, SEQ ID NO:11, SEQ ID NO:13 AND SEQ ID NO:15, or partial peptides or salts thereof.

3. The preventive/therapeutic agent according to claim 1, wherein the enzyme belonging to the acyl-CoA synthase family is at least one selected from the proteins comprising the same or substantially the same amino acid sequences represented by SEQ ID NO:1, SEQ ID NO:3, SEQ ID NO:5, SEQ ID NO:7, SEQ ID NO:9, SEQ ID NO:11, or partial peptides or salts thereof.

4. The preventive/therapeutic agent according to claim 1, wherein the fatty acid synthase is a protein comprising the same or substantially the same amino acid sequence represented by SEQ ID NO:17, or a partial peptide or salt thereof.

5. The preventive/therapeutic agent according to claim 1, prepared by combining (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family, with (ii) at least one selected from (a) a compound or salt thereof that inhibits the activity of fatty acid synthase, (b) a compound or salt thereof that inhibits expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase.

6. The preventive/therapeutic agent according to claim 1, prepared by combining a compound or salt thereof that inhibits the activity of an enzyme belonging to the acyl-CoA synthase family with a compound or salt thereof that inhibits the activity of fatty acid synthase.

7. A preventive/therapeutic agent for cancer, comprising (i) a substance that inhibits the activities of an enzyme belonging to the acyl-CoA synthase family and fatty acid synthase, and/or (ii) a substance that inhibits expression of a gene for an enzyme belonging to the acyl-CoA synthase family and expression of a fatty acid synthase gene.

8. The preventive/therapeutic agent according to claim 1, wherein the preventive/therapeutic agent for cancer is a combination preparation.

9. The preventive/therapeutic agent according to claim 1, wherein the preventive/therapeutic agent is a kit comprising (i) a medicament comprising at least one selected from a substance that inhibits the activity of an enzyme belonging to the acyl-CoA synthase family and a substance that inhibits expression of a gene for an enzyme belonging to the acyl-CoA synthase family and (ii) a medicament comprising at least one selected from a substance that inhibits the activity of fatty acid synthase and a substance that inhibits expression of a fatty acid synthase gene.

10. A method for preventing/treating cancer, wherein (i) the activity of an enzyme belonging to the acyl-CoA synthase family and/or expression of a gene for the enzyme is inhibited and (ii) the activity of fatty acid synthase and/or expression of a gene for the enzyme is inhibited.

11. A method for preventing/treating cancer, wherein (i) an effective dose of at least one selected from a substance that inhibits the activity of an enzyme belonging to the acyl-CoA synthase family and a substance that inhibits expression of a gene for an enzyme belonging to the acyl-CoA synthase family, and (ii) an effective dose of at least one selected from a substance that inhibits the activity of fatty acid synthase and a substance that inhibits expression of a fatty acid synthase gene, are administered to a mammal.

12. (canceled)

13. A cancer cell apoptosis promoting agent or cancer cell proliferation inhibiting agent prepared by combining (i) at least one selected from a substance that inhibits the activity of an enzyme belonging to the acyl-CoA synthase family and a substance that inhibits expression of a gene for an enzyme belonging to the acyl-CoA synthase family, with (ii) at least one selected from a substance that inhibits the activity of fatty acid synthase and a substance that inhibits expression of a fatty acid synthase gene.

14. The agent according to claim 13, prepared by combining (i) at least one selected from (a) a compound or salt thereof that inhibits the activity of an enzyme belonging to the acyl-CoA synthase family, (b) a compound or salt thereof that inhibits expression of a gene for an enzyme belonging to the acyl-CoA synthase family, (c) an antibody to an enzyme belonging to the acyl-CoA synthase family, (d) an antisense polynucleotide comprising a nucleotide sequence or part of a nucleotide sequence complementary or substantially complementary to the nucleotide sequence of a polynucleotide coding for an enzyme belonging to the acyl-CoA synthase family and (e) siRNA or shRNA for a polynucleotide coding for an enzyme belonging to the acyl-CoA synthase family, with (ii) at least one selected from (a) a compound or salt thereof that inhibits the activity of fatty acid synthase, (b) a compound or salt thereof that inhibits expression of a fatty acid synthase gene, (c) an antibody to fatty acid synthase, (d) an antisense polynucleotide comprising a nucleotide sequence or part of a nucleotide sequence complementary or substantially complementary to the nucleotide sequence of a polynucleotide coding for fatty acid synthase and (e) siRNA or shRNA for a polynucleotide coding for fatty acid synthase.

15. The agent according to claim 13, prepared by combining a compound or salt thereof that inhibits the activity of an enzyme belonging to the acyl-CoA synthase family with a compound or salt thereof that inhibits the activity of fatty acid synthase.

16. A cancer cell apoptosis promoting agent or cancer cell proliferation inhibiting agent, comprising (i) a substance that inhibits the activities of an enzyme belonging to the acyl-CoA synthase family and fatty acid synthase, and/or (ii) a substance that inhibits expression of a gene for an enzyme belonging to the acyl-CoA synthase family and expression of a fatty acid synthase gene.

17. A cancer cell apoptosis promotion method or cancer cell proliferation inhibition method, wherein (i) the activity of an enzyme belonging to the acyl-CoA synthase family and/or expression of a gene for the enzyme is inhibited and (ii) the activity of fatty acid synthase and/or expression of a gene for the enzyme is inhibited.


SEQUENCE LISTING (20 sequences)

SEQ ID NO: 1 - protein, 698 aa, Homo sapience
MQAHELFRYFRMPELVDFRQYVRTLPTNTLMGFGAFAALTTFWYATRPKPLKPPCDLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDAYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTLKRWLLDFASKRKEAELRSGIIRNNSLWDRLIFHKVQSSLGGRVRLMVTGAAPVSATVLTFLRAALGCQFYEGYGQTECTAGCCLTMPGDWTAGHVGAPMPCNLIKLVDVEEMNYMAAEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSTIKV

SEQ ID NO: 2 - DNA, 2094 bp, Homo sapiens
atgcaagcccatgagctgttccggtattttcgaatgccagagctggttgacttccgacagtacgtgcgtactcttccgaccaacacgcttatgggcttcggagcttttgcagcactcaccaccttctggtacgccacgagacccaaacccctgaagccgccatgcgacctctccatgcagtcagtggaagtggcgggtagtggtggtgcacgaagatccgcactacttgacagcgacgagcccttggtgtatttctatgatgatgtcacaacattatacgaaggtttccagaggggaatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggatgcctacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccacgctgaagcgatggctcttggactttgcctccaagaggaaagaagcagagcttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctgaccatgcctggagactggaccgcaggccatgttggggccccgatgccgtgcaatttgataaaacttgttgatgtggaagaaatgaattacatggctgccgagggcgagggcgaggtgtgtgtgaaagggccaaatgtatttcagggctacttgaaggacccagcgaaaacagcagaagctttggacaaagacggctggttacacacaggggacattggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccactatcaaggtt

SEQ ID NO: 3 - protein, 720 aa, Homo sapiens
MNNHVSSKPSTMKLKHTINPILLYFIHFLISLYTILTYIPFYFFSESRQEKSNRIKAKPVNSKPDSAYRSVNSLDGLASVLYPGCDTLDKVFTYAKNKFKNKRLLGTREVLNEEDEVQPNGKIFKKVILGQYNWLSYEDVFVRAFNFGNGLQMLGQKPKTNIAIFCETRAEWMIAAQACFMYNFQLVTLYATLGGPAIVHALNETEVTNIITSKELLQTKLKDIVSLVPRLRHIITVDGKPPTWSEFPKGIIVHTMAAVEALGAKASMENQPHSKPLPSDIAVIMYTSGSTGLPKGVMISHSNIIAGITGMAERIPELGEEDVYIGYLPLAHVLELSAELVCLSHGCRIGYSSPQTLADQSSKIKKGSKGDTSMLKPTLMAAVPEIMDRIYKNVMNKVSEMSSFQRNLFILAYNYKMEQISKGRNTPLCDSFVFRKVRSLLGGNIRLLLCGGAPLSATTQRFMNICFCCPVGQGYGLTESAGAGTISEVWDYNTGRVGAPLVCCEIKLKNWEEGGYFNTDKPHPRGEILIGGQSVTMGYYKNEAKTKADFFEDENGQRWLCTGDIGEFEPDGCLKIIDRKKDLVKLQAGEYVSLGKVEAALKNLPLVDNICAYANSYHSYVIGFVVPNQKELTELARKKGLKGTWEELCNSCEMENEVLKVLSEAAISASLEKFEIPVKIRLSPEPWTPETGLVTDAFKLKRKELKTHYQADIERMYGRK

SEQ ID NO: 4 - DNA, 2160 bp, Homo sapiens
atgaataaccacgtgtcttcaaaaccatctaccatgaagctaaaacataccatcaaccctattcttttatattttatacattttctaatatcactttatactattttaacatacattccgttttattttttctccgagtcaagacaagaaaaatcaaaccgaattaaagcaaagcctgtaaattcaaaacctgattctgcatacagatctgttaatagtttggatggtttggcttcagtattataccctggatgtgatactttagataaagtttttacatatgcaaaaaacaaatttaagaacaaaagactcttgggaacacgtgaagttttaaatgaggaagatgaagtacaaccaaatggaaaaatttttaaaaaggttattcttggacagtataattggctttcctatgaagatgtctttgttcgagcctttaattttggaaatggattacagatgttgggtcagaaaccaaagaccaacatcgccatcttctgtgagaccagggccgagtggatgatagctgcacaggcgtgttttatgtataattttcagcttgttacattatatgccactctaggaggtccagccattgttcatgcattaaatgaaacagaggtgaccaacatcattactagtaaagaactcttacaaacaaagttgaaggatatagtttctttggtcccacgcctgcggcacatcatcactgttgatggaaagccaccgacctggtccgagttccccaagggcatcattgtgcataccatggctgcagtggaggccctgggagccaaggccagcatggaaaaccaacctcatagcaaaccattgccctcagatattgcagtaatcatgtacacaagtggatccacaggacttccaaagggagtcatgatctcacatagtaacattattgctggtataactgggatggcagaaaggattccagaactaggagaggaagatgtctacattggatatttgcctctggcccatgttctagaattaagtgctgagcttgtctgtctttctcacggatgccgcattggttactcttcaccacagactttagcagatcagtcttcaaaaattaaaaaaggaagcaaaggggatacatccatgttgaaaccaacactgatggcagcagttccggaaatcatggatcggatctacaaaaatgtcatgaataaagtcagtgaaatgagtagttttcaacgtaatctgtttattctggcctataattacaaaatggaacagatttcaaaaggacgtaatactccactgtgcgacagctttgttttccggaaagttcgaagcttgctagggggaaatattcgtctcctgttgtgtggtggcgctccactttctgcaaccacgcagcgattcatgaacatctgtttctgctgtcctgttggtcagggatacgggctcactgaatctgctggggctggaacaatttccgaagtgtgggactacaatactggcagagtgggagcaccattagtttgctgtgaaatcaaattaaaaaactgggaggaaggtggatactttaatactgataagccacaccccaggggtgaaattcttattgggggccaaagtgtgacaatggggtactacaaaaatgaagcaaaaacaaaagctgatttctttgaagatgaaaatggacaaaggtggctctgtactggggatattggagagtttgaacccgatggatgcttaaagattattgatcgtaaaaaggaccttgtaaaactacaggcaggggaatatgtttctcttgggaaagtagaggcagctttgaagaatcttccactagtagataacatttgtgcatatgcaaacagttatcattcttatgtcattggatttgttgtgccaaatcaaaaggaactaactgaactagctcgaaagaaaggacttaaagggacttgggaggagctgtgtaacagttgtgaaatggaaaatgaggtacttaaagtgctttccgaagctgctatttcagcaagtctggaaaagtttgaaattccagtaaaaattcgtttgagtcctgaaccgtggacccctgaaactggtctggtgacagatgccttcaagctgaaacgcaaagagcttaaaacacattaccaggcggacattgagcgaatgtatggaagaaaa

SEQ ID NO: 5 - protein, 670 aa, Homo sapiens
MAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 6 - DNA, 2010 bp, Homo sapiens
atggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaa

SEQ ID NO: 7 - protein, 711 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 8 - DNA, 2133 bp, Homo sapiens
atgaaacttaagctaaatgtgctcaccattattttgctgcctgtccacttgttaataacaatatacagtgcccttatatttattccatggtattttcttaccaatgccaagaagaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaa

SEQ ID NO: 9 - protein, 739 aa, Homo sapiens
MDALKPPCLWRNHERGKKDRDSCGRKNSEPGSPHSLEALRDAAPSQGLNFLLLFTKMLFIFNFLFSPLPTPALICILTFGAAIFLWLITRPQPVLPLLDLNNQSVGIEGGARKGVSQKNNDLTSCCFSDAKTMYEVFQRGLAVSDNGPCLGYRKPNQPYRWLSYKQVSDRAEYLGSCLLHKGYKSSPDQFVGIFAQNRPEWIISELACYTYSMVAVPLYDTLGPEAIVHIVNKADIAMVICDTPQKALVLIGNVEKGFTPSLKVIILMDPFDDDLKQRGEKSGIEILSLYDAENLGKEHFRKPVPPSPEDLSVICFTSGTTGDPKGAMITHQNIVSNAAAFLKCVEHAYEPTPDDVAISYLPLAHMFERIVQAVVYSCGARVGFFQGDIRLLADDMKTLKPTLFPAVPRLLNRIYDKVQNEAKTPLKKFLLKLAVSSKFKELQKGIIRHDSFWDKLIFAKIQDSLGGRVRVIVTGAAPMSTSVMTFFRAAMGCQVYEAYGQTECTGGCTFTLPGDWTSGHVGVPLACNYVKLEDVADMNYFTVNNEGEVCIKGTNVFKGYLKDPEKTQEALDSDGWLHTGDIGRWLPNGTLKIIDRKKNIFKLAQGEYIAPEKIENIYNRSQPVLQIFVHGESLRSSLVGVVVPDTDVLPSFAAKLGVKGSFEELCQNQVVREAILEDLQKIGKESGLKTFEQVKAIFLHPEPFSIENGLLTPTLKAKRGELSKYFRTQIDSLYEHIQD

SEQ ID NO: 10 - DNA, 2217 bp, Homo sapiens
atggacgctctgaagccaccctgtctctggaggaaccacgagcgagggaagaaggacagggactcgtgtggcaggaagaactcagagccgggaagcccccattcactagaagcactgagagatgcggccccctcgcagggtctgaatttcctgctgctgttcacaaagatgctttttatctttaactttttgttttccccacttccgaccccggcgttgatctgcatcctgacatttggagctgccatcttcttgtggctgatcaccagacctcaacccgtcttacctcttcttgacctgaacaatcagtctgtgggaattgagggaggagcacggaagggggtttcccagaagaacaatgacctaacaagttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacacgtactctatggtagctgtacctctgtatgacaccttgggaccagaagccatcgtacatattgtcaacaaggctgatatcgccatggtgatctgtgacacaccccaaaaggcattggtgctgatagggaatgtagagaaaggcttcaccccgagcctgaaggtgatcatccttatggacccctttgatgatgacctgaagcaaagaggggagaagagtggaattgagatcttatccctatatgatgctgagaacctaggcaaagagcacttcagaaaacctgtgcctcctagcccagaagacctgagcgtcatctgcttcaccagtgggaccacaggtgaccccaaaggagccatgataacccatcaaaatattgtttcaaatgctgctgcctttctcaaatgtgtggagcatgcttatgagcccactcctgatgatgtggccatatcctacctccctctggctcatatgtttgagaggattgtacaggctgttgtgtacagctgtggagccagagttggattcttccaaggggatattcggttgctggctgacgacatgaagactttgaagcccacattgtttcccgcggtgcctcgactccttaacaggatctacgataaggtacaaaatgaggccaagacacccttgaagaagttcttgttgaagctggctgtttccagtaaattcaaagagcttcaaaagggtatcatcaggcatgatagtttctgggacaagctcatctttgcaaagatccaggacagcctgggcggaagggttcgtgtaattgtcactggagctgcccccatgtccacttcagtcatgacattcttccgggcagcaatgggatgtcaggtgtatgaagcttatggtcaaacagaatgcacaggtggctgtacatttacattacctggggactggacatcaggtcacgttggggtgcccctggcttgcaattacgtgaagctggaagatgtggctgacatgaactactttacagtgaataatgaaggagaggtctgcatcaagggtacaaacgtgttcaaaggatacctgaaggaccctgagaagacacaggaagccctggacagtgatggctggcttcacacaggagacattggtcgctggctcccgaatggaactctgaagatcatcgaccgtaaaaagaacattttcaagctggcccaaggagaatacattgcaccagagaagatagaaaatatctacaacaggagtcaaccagtgttacaaatttttgtacacggggagagcttacggtcatccttagtaggagtggtggttcctgacacagatgtacttccctcatttgcagccaagcttggggtgaagggctcctttgaggaactgtgccaaaaccaagttgtaagggaagccattttagaagacttgcagaaaattgggaaagaaagtggccttaaaacttttgaacaggtcaaagccatttttcttcatccagagccattttccattgaaaatgggctcttgacaccaacattgaaagcaaagcgaggagagctttccaaatactttcggacccaaattgacagcctgtatgagcacatccaggat

SEQ ID NO: 11 - protein, 683 aa, Homo sapiens
MLFIFNFLFSPLPTPALICILTFGAAIFLWLITRPQPVLPLLDLNNQSVGIEGGARKGVSQKNNDLTSCCFSDAKTMYEVFQRGLAVSDNGPCLGYRKPNQPYRWLSYKQVSDRAEYLGSCLLHKGYKSSPDQFVGIFAQNRPEWIISELACYTYSMVAVPLYDTLGPEAIVHIVNKADIAMVICDTPQKALVLIGNVEKGFTPSLKVIILMDPFDDDLKQRGEKSGIEILSLYDAENLGKEHFRKPVPPSPEDLSVICFTSGTTGDPKGAMITHQNIVSNAAAFLKCVEHAYEPTPDDVAISYLPLAHMFERIVQAVVYSCGARVGFFQGDIRLLADDMKTLKPTLFPAVPRLLNRIYDKVQNEAKTPLKKFLLKLAVSSKFKELQKGIIRHDSFWDKLIFAKIQDSLGGRVRVIVTGAAPMSTSVMTFFRAAMGCQVYEAYGQTECTGGCTFTLPGDWTSGHVGVPLACNYVKLEDVADMNYFTVNNEGEVCIKGTNVFKGYLKDPEKTQEALDSDGWLHTGDIGRWLPNGTLKIIDRKKNIFKLAQGEYIAPEKIENIYNRSQPVLQIFVHGESLRSSLVGVVVPDTDVLPSFAAKLGVKGSFEELCQNQVVREAILEDLQKIGKESGLKTFEQVKAIFLHPEPFSIENGLLTPTLKAKRGELSKYFRTQIDSLYEHIQD

SEQ ID NO: 12 - DNA, 2049 bp, Homo sapiens
atgctttttatctttaactttttgttttccccacttccgaccccggcgttgatctgcatcctgacatttggagctgccatcttcttgtggctgatcaccagacctcaacccgtcttacctcttcttgacctgaacaatcagtctgtgggaattgagggaggagcacggaagggggtttcccagaagaacaatgacctaacaagttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacacgtactctatggtagctgtacctctgtatgacaccttgggaccagaagccatcgtacatattgtcaacaaggctgatatcgccatggtgatctgtgacacaccccaaaaggcattggtgctgatagggaatgtagagaaaggcttcaccccgagcctgaaggtgatcatccttatggacccctttgatgatgacctgaagcaaagaggggagaagagtggaattgagatcttatccctatatgatgctgagaacctaggcaaagagcacttcagaaaacctgtgcctcctagcccagaagacctgagcgtcatctgcttcaccagtgggaccacaggtgaccccaaaggagccatgataacccatcaaaatattgtttcaaatgctgctgcctttctcaaatgtgtggagcatgcttatgagcccactcctgatgatgtggccatatcctacctccctctggctcatatgtttgagaggattgtacaggctgttgtgtacagctgtggagccagagttggattcttccaaggggatattcggttgctggctgacgacatgaagactttgaagcccacattgtttcccgcggtgcctcgactccttaacaggatctacgataaggtacaaaatgaggccaagacacccttgaagaagttcttgttgaagctggctgtttccagtaaattcaaagagcttcaaaagggtatcatcaggcatgatagtttctgggacaagctcatctttgcaaagatccaggacagcctgggcggaagggttcgtgtaattgtcactggagctgcccccatgtccacttcagtcatgacattcttccgggcagcaatgggatgtcaggtgtatgaagcttatggtcaaacagaatgcacaggtggctgtacatttacattacctggggactggacatcaggtcacgttggggtgcccctggcttgcaattacgtgaagctggaagatgtggctgacatgaactactttacagtgaataatgaaggagaggtctgcatcaagggtacaaacgtgttcaaaggatacctgaaggaccctgagaagacacaggaagccctggacagtgatggctggcttcacacaggagacattggtcgctggctcccgaatggaactctgaagatcatcgaccgtaaaaagaacattttcaagctggcccaaggagaatacattgcaccagagaagatagaaaatatctacaacaggagtcaaccagtgttacaaatttttgtacacggggagagcttacggtcatccttagtaggagtggtggttcctgacacagatgtacttccctcatttgcagccaagcttggggtgaagggctcctttgaggaactgtgccaaaaccaagttgtaagggaagccattttagaagacttgcagaaaattgggaaagaaagtggccttaaaacttttgaacaggtcaaagccatttttcttcatccagagccattttccattgaaaatgggctcttgacaccaacattgaaagcaaagcgaggagagctttccaaatactttcggacccaaattgacagcctgtatgagcacatccaggat

SEQ ID NO: 13 - protein, 722 aa, Homo sapiens
MLTFFLVSGGSLWLFVEFVLSLLEKMQTQEILRILRLPELGDLGQFFRSLSATTLVSMGALAAILAYWFTHRPKALQPPCNLLMQSEEVEDSGGARRSVIGSGPQLLTHYYDDARTMYQVFRRGLSISGNGPCLGFRKPKQPYQWLSYQEVADRAEFLGSGLLQHNCKACTDQFIGVFAQNRPEWIIVELACYTYSMVVVPLYDTLGPGAIRYIINTADISTVIVDKPQKAVLLLEHVERKETPGLKLIILMDPFEEALKERGQKCGVVIKSMQAVEDCGQENHQAPVPPQPDDLSIVCFTSGTTGNPKGAMLTHGNVVADFSGFLKVTESQWAPTCADVHISYLPLAHMFERMVQSVVYCHGGRVGFFQGDIRLLSDDMKALCPTIFPVVPRLLNRMYDKIFSQANTPLKRWLLEFAAKRKQAEVRSGIIRNDSIWDELFFNKIQASLGGCVRMIVTGAAPASPTVLGFLRAALGCQVYEGYGQTECTAGCTFTTPGDWTSGHVGAPLPCNHIKLVDVEELNYWACKGEGEICVRGPNVFKGYLKDPDRTKEALDSDGWLHTGDIGKWLPAGTLKIIDRKKHIFKLAQGEYVAPEKIENIYIRSQPVAQIYVHGDSLKAFLVGIVVPDPEVMPSWAQKRGIEGTYADLCTNKDLKKAILEDMVRLGKESGLHSFEQVKAIHIHSDMFSVQNGLLTPTLKAKRPELREYFKKQIEELYSISM

SEQ ID NO: 14 - DNA, 2166 bp, Homo sapiens
atgctgaccttcttcctcgtgtcggggggctccctctggctattcgtagagtttgtcctctcacttctggagaagatgcagacacaggagatcctgaggatactgcgactgcctgagctaggtgacttgggacagtttttccgcagcctctcggccaccaccctcgtgagtatgggtgccctggctgccatccttgcctactggttcactcaccggccaaaggccttgcagccgccatgcaacctcctgatgcagtcagaagaagtagaggacagtggcggggcacggcgatctgtgattgggtctggccctcagctacttacccactactatgatgatgcccggaccatgtaccaggtgttccgccgtgggcttagcatctcagggaatgggccctgtcttggtttcaggaagcctaagcagccttaccagtggctgtcctaccaggaggtggccgacagggctgaatttctggggtccggacttctccagcacaattgtaaagcatgcactgatcagtttattggtgtttttgcacaaaatcggccagagtggatcattgtggagctggcctgctacacatattccatggtggtggtcccgctctatgacaccctgggccctggggctatccgctacatcatcaatacagcggacatcagcaccgtgattgtggacaaacctcagaaggctgtgcttctgctagagcatgtggagaggaaggagactccaggcctcaagctgatcatcctcatggacccattcgaagaagccctgaaagagagagggcagaagtgcggggtggtcattaagtccatgcaggccgtggaggactgtggccaagagaatcaccaggctcctgtgcccccgcagcctgatgacctctccattgtgtgtttcacaagcggcacgacagggaacccaaaaggtgcgatgctcacccatgggaacgtggtggctgatttctcaggctttctgaaagtgacagagagtcagtgggctcccacttgtgcggatgtgcacatttcctatttgcctttagcacacatgtttgagcgaatggtgcagtctgtcgtctattgccacggagggcgtgttggcttcttccagggagatatccgccttctctcagatgacatgaaggctctatgccccaccatcttccctgtggtcccacgactgctgaaccggatgtacgacaagatcttcagccaggcaaacacaccattaaagcgctggctcctggagtttgcagcaaagcgtaagcaagccgaggtccggagtggaatcatcaggaatgatagtatctgggatgaactcttctttaataagattcaggccagtcttggtgggtgtgtgcggatgattgttactggagcagccccagcatcaccaacagttctgggatttctccgggcagctctagggtgccaggtttatgaaggttatggccaaactgagtgcacagctggatgtaccttcaccactcctggcgactggacctcagggcacgtaggggcgccacttccctgcaatcatatcaagctcgttgatgttgaggaactgaactactgggcctgcaaaggagagggagagatatgtgtgagaggaccaaatgtgttcaaaggctacttgaaagatccagacaggacgaaggaggccctggacagcgatggctggcttcacactggagacatcggaaaatggctgccggcaggaactcttaaaattattgatcggaaaaagcatatatttaaacttgctcagggagaatatgttgcacccgagaagattgagaacatctacatccggagccaacctgtggcgcaaatctatgtccatggggacagcttaaaggcctttttggtaggcattgttgtgcctgaccctgaagttatgccctcctgggcccagaagagaggaattgaaggaacatatgcagatctctgcacaaataaggatctgaagaaagccattttggaagatatggtgaggttaggaaaagaaagtggactccattcttttgagcaggttaaagccattcacatccattctgacatgttctcagttcaaaatggcttgctgacaccaacactaaaagctaagagacctgagctgagagagtacttcaaaaaacaaatagaagagctttactcaatctccatg

SEQ ID NO: 15 - protein, 722 aa, Homo sapiens
MLTFFLVSGGSLWLFVEFVLSLLEKMQTQEILRILRLPELGDLGQFFRSLSATTLVSMGALAAILAYWFTHRPKALQPPCNLLMQSEEVEDSGGARRSVIGSGPQLLTHYYDDARTMYQVFRRGLSISGNGPCLGFRKPKQPYQWLSYQEVADRAEFLGSGLLQHNCKACTDQFIGVFAQNRPEWIIVELACYTYSMVVVPLYDTLGPGAIRYIINTADISTVIVDKPQKAVLLLEHVERKETPGLKLIILMDPFEEALKERGQKCGVVIKSMQAVEDCGQENHQAPVPPQPDDLSIVCFTSGTTGNPKGAMLTHGNVVADFSGFLKVTEKVIFPRQDDVLISFLPLAHMFERVIQSVVYCHGGRVGFFQGDIRLLSDDMKALCPTIFPVVPRLLNRMYDKIFSQANTPLKRWLLEFAAKRKQAEVRSGIIRNDSIWDELFFNKIQASLGGCVRMIVTGAAPASPTVLGFLRAALGCQVYEGYGQTECTAGCTFTTPGDWTSGHVGAPLPCNHIKLVDVEELNYWACKGEGEICVRGPNVFKGYLKDPDRTKEALDSDGWLHTGDIGKWLPAGTLKIIDRKKHIFKLAQGEYVAPEKIENIYIRSQPVAQIYVHGDSLKAFLVGIVVPDPEVMPSWAQKRGIEGTYADLCTNKDLKKAILEDMVRLGKESGLHSFEQVKAIHIHSDMFSVQNGLLTPTLKAKRPELREYFKKQIEELYSISM

SEQ ID NO: 16 - DNA, 2166 bp, Homo sapiens
atgctgaccttcttcctcgtgtcggggggctccctctggctattcgtagagtttgtcctctcacttctggagaagatgcagacacaggagatcctgaggatactgcgactgcctgagctaggtgacttgggacagtttttccgcagcctctcggccaccaccctcgtgagtatgggtgccctggctgccatccttgcctactggttcactcaccggccaaaggccttgcagccgccatgcaacctcctgatgcagtcagaagaagtagaggacagtggcggggcacggcgatctgtgattgggtctggccctcagctacttacccactactatgatgatgcccggaccatgtaccaggtgttccgccgtgggcttagcatctcagggaatgggccctgtcttggtttcaggaagcctaagcagccttaccagtggctgtcctaccaggaggtggccgacagggctgaatttctggggtccggacttctccagcacaattgtaaagcatgcactgatcagtttattggtgtttttgcacaaaatcggccagagtggatcattgtggagctggcctgctacacatattccatggtggtggtcccgctctatgacaccctgggccctggggctatccgctacatcatcaatacagcggacatcagcaccgtgattgtggacaaacctcagaaggctgtgcttctgctagagcatgtggagaggaaggagactccaggcctcaagctgatcatcctcatggacccattcgaagaagccctgaaagagagagggcagaagtgcggggtggtcattaagtccatgcaggccgtggaggactgtggccaagagaatcaccaggctcctgtgcccccgcagcctgatgacctctccattgtgtgtttcacaagcggcacgacagggaacccaaaaggtgcgatgctcacccatgggaacgtggtggctgatttctcaggctttctgaaagtgacagagaaagtgatctttccgagacaggacgatgtgctcatctccttcctgcctctggctcacatgtttgagagagtaatccagtctgtcgtctattgccacggagggcgtgttggcttcttccagggagatatccgccttctctcagatgacatgaaggctctatgccccaccatcttccctgtggtcccacgactgctgaaccggatgtacgacaagatcttcagccaggcaaacacaccattaaagcgctggctcctggagtttgcagcaaagcgtaagcaagccgaggtccggagtggaatcatcaggaatgatagtatctgggatgaactcttctttaataagattcaggccagtcttggtgggtgtgtgcggatgattgttactggagcagccccagcatcaccaacagttctgggatttctccgggcagctctagggtgccaggtttatgaaggttatggccaaactgagtgcacagctggatgtaccttcaccactcctggcgactggacctcagggcacgtaggggcgccacttccctgcaatcatatcaagctcgttgatgttgaggaactgaactactgggcctgcaaaggagagggagagatatgtgtgagaggaccaaatgtgttcaaaggctacttgaaagatccagacaggacgaaggaggccctggacagcgatggctggcttcacactggagacatcggaaaatggctgccggcaggaactcttaaaattattgatcggaaaaagcatatatttaaacttgctcagggagaatatgttgcacccgagaagattgagaacatctacatccggagccaacctgtggcgcaaatctatgtccatggggacagcttaaaggcctttttggtaggcattgttgtgcctgaccctgaagttatgccctcctgggcccagaagagaggaattgaaggaacatatgcagatctctgcacaaataaggatctgaagaaagccattttggaagatatggtgaggttaggaaaagaaagtggactccattcttttgagcaggttaaagccattcacatccattctgacatgttctcagttcaaaatggcttgctgacaccaacactaaaagctaagagacctgagctgagagagtacttcaaaaaacaaatagaagagctttactcaatctccatg

SEQ ID NO: 17 - protein, 2511 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

SEQ ID NO: 18 - DNA, 7533 bp, Homo sapiens
atggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggc

SEQ ID NO: 19 - DNA, 41 bp, Artificial Sequence
  Primer
aaagaattctatgctttttatctttaactttttgttttccc

SEQ ID NO: 20 - DNA, 35 bp, Artificial Sequence
  Primer
aaaggatccataatcctggatgtgctcatacaggc